(19) United States
(12) Patent Application Publication
(10) Pub. No.: US 2023/0357760 A1
(43) Pub. Date: Nov. 9, 2023

(54) METHOD AND AGENT FOR TREATING/PREVENTING NEURODEGENERATIVE DISEASE AND ASSOCIATED NEUROINFLAMMATION AND FOR EVALUATING PUTATIVE PROPHYLACTICS/THERAPEUTICS FOR TREATING/PREVENTING NEURODEGENERATIVE DISEASE AND NEUROINFLAMMATION

(71) Applicant: THE TRUSTEES OF DARTMOUTH COLLEGE, Hanover, NH (US)

(72) Inventors: Matthew C. HAVRDA, Etna, NH (US); Owen M. WILKINS, Lebanon, NH (US)

(21) Appl. No.: 18/029,717

(22) PCT Filed: Sep. 30, 2021
(86) PCT No.: PCT/US2021/052964

§ 371 Date: Mar. 31, 2023

Related U.S. Application Data
(60) Provisional application No. 63/086,765, filed on Oct. 2, 2020.

(51) Int. Cl. C12N 15/11; C12N 9/22; A61K 45/06; A61K 31/7088; A61K 38/46; C12Q 1/6883; G01N 33/68; G01N 33/50

(52) U.S. Cl. CPC C12N 15/11; C12N 9/22; A61K 45/06; A61K 31/7088; A61K 38/465; C12Q 1/6883; G01N 33/6896; G01N 33/5023; C12N 2310/20; G01N 2800/56; G01N 2800/2835; G01N 2800/52; C12Q 2600/158; G01N 2800/60; C12Q 2600/106; C12Q 2600/112

(57) ABSTRACT

The present invention relates to methods of treating and/or preventing or slowing the onset of neurodegenerative diseases such as Parkinson's disease (PD), methods of treating and/or preventing or slowing the onset of inflammation associated with neurodegenerative diseases such as PD, methods of diagnosing neurodegenerative diseases such as PD, methods of determining the severity and/or stage of neurodegenerative diseases such as PD, methods of determining the inflammatory status or levels in neurodegenerative diseases such as PD, methods of determining whether a therapy for neurodegenerative diseases such as PD is effective in a subject, methods of screening for a therapeutic agent for neurodegenerative diseases such as PD, and methods of predicting whether a subject with a neurodegenerative disease such as PD will respond to a therapy. The present invention further relates to agents that suppress, inhibit, block, and/or antagonize BRI3, compositions comprising such an agent, and kits for detecting expression of BRI3.

This application claims the priority benefit of U.S. Provisional Application Ser. No. 63/086,765 filed Oct. 2, 2020, which is incorporated by reference herein in their entirety.

### FIELD OF THE INVENTION

The present invention relates to methods of treating and/or preventing and/or delaying the onset of neurodegenerative diseases such as Parkinson's disease (PD), methods of treating and/or preventing neural inflammation associated with neurodegenerative diseases such as PD, methods of diagnosing neurodegenerative diseases such as PD, methods of determining the severity and/or stage of neurodegenerative diseases such as PD, methods of determining the inflammatory status or levels in neurodegenerative diseases such as PD, methods of determining whether a therapy for neurodegenerative diseases such as PD is effective in a subject, methods of screening for a therapeutic agent for neurodegenerative diseases such as PD, and methods of predicting whether a subject with a neurodegenerative disease such as PD will respond to a therapy. The present invention further relates to agents that suppress, inhibit, block, and/or antagonize BRI3, compositions comprising such an agent, and kits for detecting expression of BRI3.

### BACKGROUND OF THE INVENTION

Parkinson's disease (PD) is a slowly progressive age-related neurodegenerative disorder characterized by motor impairments and a host of non-motor symptomatology. An infrequently detected prodromal period combined with the sometimes decades long progression of PD make it difficult to pinpoint disease initiation, impeding the development of preventative and curative therapies. Practical hurdles exist in the sampling of biofluids and tissues from the central nervous system of PD patients. There is growing appreciation of the role of the immune system in the progression of PD. Inflammation is a characteristic of PD likely triggered by non-microbial danger associated molecular patterns including environmental toxicants, oxidative stress, protein misfolding, and cell death.

### BRIEF DESCRIPTION OF THE FIGURES

FIG. 1A-D. Peripheral blood mononuclear cells (PBMCs) from five volunteers [n=2 healthy control (3401HC and 3402HC), 3 PD (PD134, 123, 126)] were subject to scRNASeq and the entire dataset was integrated to examine cell-type specific changes in gene expression in PD. Panel A contains a schematic representing the overall workflow from patient sampling to identification of differentially expressed genes in specific cell types (black arrows represent work flow), (UMAP: Uniform Manifold Approximation and Projection, SCINA: Semi-supervised Category Identification and Assignment, Muskat: Multi-sample multi-group scRNA-seq analysis tools). Panel B shows a table indicating the totals and percentages of various cell types in PD patients and controls. Panel C shows that the data integration pipeline successfully normalized the dataset to eliminate subject-driven variability. Panel D shows that no group-based variability was noted in the identification of clusters or assignment of cellular identity.

FIG. 2A-B contain experimental results relating to the identification of differentially expressed genes (DEGs) in monocytes. Panel A contains an MA plot demonstrating genes significantly altered in CD16 monocytes in PD [P value: 1.00e-11(p_adj_loc)]. Panel B contains a Dot plot indicating percent detection (dot size) of DEGs split in order to compare PD patients (blue) to healthy controls (red). The data indicate that BRI3 expression levels are significantly elevated in PD (A), BRI3 expression is relatively restricted to monocytes (B), and that BRI3 is detectable in a higher percentage of PD patient monocytes compared to controls (B).

FIG. 3 contains a data summary of the results of experiments which determined the frequency and consistency of cells driving DEG findings. Volcano plots of selected genes demonstrating variation in individual patients and number of cells in which DEGs were detected are shown. The data shows that BRI3 elevation was consistently elevated in significant numbers of cells in all three PD patients sampled compared to control subjects (upper middle).

FIG. 4A-B contain experimental results wherein BRI3 was induced using inflammatory stimuli in a monocyte cell line. Panel A contains a Western blot demonstrating BRI3 upregulation and release following activation of cultured human THP-1 monocytes activated using sequential exposure to LPS and nigericin. Panel B shows the quantitation of independent western blot experiments using densitometry (n=3, P value>0.05, t-test).

FIG. 5A-D contain experimental results demonstrating that the inactivation of BRI3 reduces cytokine expression. Panel A contains a Western blot analysis of four independent sgRNAs targeting BRI3. The information in the box depicts loss of BRI3 protein in cell lines derived using two independent sgRNAs (C and D). Panel B contains experimental results showing the confirmation of sequence alterations resulting from CRISPR/CAS9 inactivation of BRI3 genomic sequence in THP1 cells. Panel C and D contain experimental results showing the suppression of G-CSF (C) and IL2 (D) in THP-1 cells lacking BRI3 following sequential exposure to LPS and nigericin.

### OBJECTS OF THE INVENTION

It is an object of the invention to provide novel methods of determining whether a subject has a neurodegenerative disease associated with neuroinflammation or whether a subject is at increased risk of developing a neurodegenerative disease associated with neuroinflammation.

It is an another object of the invention to provide novel methods of treating or preventing or inhibiting the onset of a neurodegenerative disease associated with neuroinflammation.

It is a specific object of the invention to provide novel methods of treating or preventing or inhibiting the onset of a neurodegenerative disease associated with neuroinflammation, comprising administering to a subject in need thereof an active agent that modifies the expression and/or function of BRI3, FAM89B, PCBP1, ATP5F1E (or ATP5E), SH2B2, LMO2, TAF10, MAP2K2, TLE4, GRK6, RNF187, TNNT1, PTP4A2, SIAH2, YBX3, LAMP1, RPL8, EID2, CAPG, SRM, TMSB10, FYB, HLA-E, GLRX5, SEC61G, TMEM9, DBP, XRRA1, BLOC1S4, TUFM, HDDC2, EVL, UBB, RAC2, OAS1, LSP1, or ARF5, or any combination thereof, optionally wherein at least the expression and/or function of BRI3 is modified (increased) in the subject, optionally wherein the neurodegenerative disease associated with neuroinflammation is Parkinson's disease (PD).

It is another specific object of the invention to provide novel methods of treating or preventing or inhibiting the onset of neural inflammation associated with a neurodegenerative disease, comprising administering to a subject in need thereof an active agent that modifies the expression and/or function of BRI3, FAM89B, PCBP1, ATP5F1E (or ATP5E), SH2B2, LMO2, TAF10, MAP2K2, TLE4, GRK6, RNF187, TNNT1, PTP4A2, SIAH2, YBX3, LAMP1, RPL8, EID2, CAPG, SRM, TMSB10, FYB, HLA-E, GLRX5, SEC61G, TMEM9, DBP, XRRA1, BLOC1S4, TUFM, HDDC2, EVL, UBB, RAC2, OAS1, LSP1, or ARF5, or any combination thereof, optionally wherein at least the expression and/or function of BRI3 is modified.

It is another specific object of the invention to provide novel methods of treatment or prevention as above, wherein the active agent reduces the expression and/or function of BRI3, FAM89B, PCBP1, ATP5F1E (or ATP5E), SH2B2, LMO2, TAF10, MAP2K2, TLE4, GRK6, RNF187, TNNT1, PTP4A2, SIAH2, YBX3, LAMP1, RPL8, EID2, CAPG, SRM, TMSB10, FYB, HLA-E, GLRX5, SEC61G, TMEM9, DBP, XRRA1, BLOC1S4, or TUFM, or any combination thereof, optionally wherein at least the expression and/or function of BRI3 is reduced, further optionally wherein the reduction is by about 25%, about 30%, about 40%, about 45%, about 50%, about 55% about 60%, about 65%, about 70%, about 75%, about 80%, about 85%, about 90%, about 95%, or about 100%.

It is another specific object of the invention to provide novel methods of treatment or prevention as above, wherein the active agent increases the expression and/or function of HDDC2, EVL, UBB, RAC2, OAS1, LSP1, or ARF5, or any combination thereof, optionally wherein the increase is by about 25%, about 30%, about 40%, about 45%, about 50%, about 55% about 60%, about 65%, about 70%, about 75%, about 80%, about 85%, about 90%, about 95%, about 100%, about 150%, about 200%, about 300%, about 400%, about 500%, about 600%, about 800%, about 900%, about 1000%, about 2000%, about 3000%, about 4000%, about 5000%, or about 10000%.

It is another specific object of the invention to provide novel methods of treatment or prevention as above, wherein the modification of gene expression or function takes place at least in peripheral blood mononuclear cells (PBMCs), monocytes, dendritic cells, and/or central nervous system (CNS) cells, optionally wherein the monocytes are circulating monocytes, further optionally wherein the monocytes are CD16⁺ monocytes and/or CD14⁺ monocytes, and yet further optionally wherein the CNS cells comprise or are microglia.

It is another specific object of the invention to provide novel methods of treatment or prevention as above, wherein the active agent at least reduces the expression and/or function of BRI3 at least in monocytes, optionally wherein the monocytes are circulating monocytes, further optionally wherein the monocytes are CD16⁺ monocytes CD14⁺ monocytes, and/or meningeal monocytes.

It is another specific object of the invention to provide novel methods of treatment or prevention as above, wherein the active agent that modifies the expression and/or function comprises a clustered Regularly Interspaced Short Palindromic Repeat (CRISPR)/Cas gene editing agent, a zinc-finger nuclease (ZFN) gene editing agent, a transcription activator-like effector nuclease (TALEN) gene editing agent, a transposase-based gene therapy, an siRNA, an shRNA, an miRNA, an aptamer, an antibody, an antigen-binding antibody fragment (e.g., scFv, Fab, Fab′, (Fab′)2), a chimeric antigen receptor (CAR)-expressing cell, a peptide, a small molecule, a polymer, an expression vector encoding a gene of interest, or any combination thereof,

It is another specific object of the invention to provide novel methods of treatment or prevention as above, wherein the active agent that modifies the expression and/or function comprises a clustered Regularly Interspaced Short Palindromic Repeat (CRISPR)/Cas gene editing agent, wherein the active agent comprises or consists of one or more of the following:
(i) a CRISPR/Cas gene editing agent against BRI3;
(ii) a short-guide RNA (sgRNA) selected from the oligonucleotide sequences AACTCTATCGTGGTCGTAGG, CGTCACAGGTGGGCCCGTAA, GACTACGCGTGCGGCCCGCA (depicted here in “sense” orientation),
(iii) a sgRNA targeting BRI3 falling within or including any of the following genomic sequences: GAGGAAGCGACGATGCCCCAACTGTGGAGC, ACCACGATAGAGTTGGCAGGATAGCGGGTG, AGTTGGGGCATCGTCGCTTCCTCAAGGCAA, TTACGGGCCCACCTGTGACGAGGTAGGGGT, GCCCTACCCCTACCTCGTCACAGGTGGGCC, TCCTGCCAACTCTATCGTGGTCGTAGGAGG, CCCGCTATCCTGCCAACTCTATCGTGGTCG, GGGCGACTACGCGTGCGGCCCGCACGGCTA, GCCCACCTGTGACGAGGTAGGGGTAGGGCG, CCCAGGGTCTACAACATCCACAGCCGGACC, CCACGATAGAGTTGGCAGGATAGCGGGTGA, TTGGCAGGATAGCGGGTGACGGTCCGGCTG, CAGGGATACCCACCCACCATCCCAGGGTCT, CCTGGTGTTCCCTTTAAGCGAAGGTGGCTC (as annotated in Gene ID 25798, NM_015379.5, or identical BRI3 sequences in prior or future annotations),
(iv) a sgRNA sequence selected from one comprising or consisting of any of the following nucleic acid sequences:

GAGGAAGCGACGATGCCCCAACTGTGGAGC
GAGGAAGCGACGATGCCCCAACTGTGGAGC
GAGGAAGCGACGATGCCCCAACTGTGGAGC
GAGGAAGCGACGATGCCCCAACTGTGGAGC
GAGGAAGCGACGATGCCCCAACTGTGGAGC
GAGGAAGCGACGATGCCCCAACTGTGGAGC
GAGGAAGCGACGATGCCCCAACTGTGGAGC
GAGGAAGCGACGATGCCCCAACTGTGGAGC
GAGGAAGCGACGATGCCCCAACTGTGGAGC
GAGGAAGCGACGATGCCCCAACTGTGGAGC
GAGGAAGCGACGATGCCCCAACTGTGGAGC
ACCACGATAGAGTTGGCAGGATAGCGGGTG
AGTTGGGGCATCGTCGCTTCCTCAAGGCAA
TTACGGGCCCACCTGTGACGAGGTAGGGGT
GCCCTACCCCTACCTCGTCACAGGTGGGCC
TCCTGCCAACTCTATCGTGGTCGTAGGAGG
CCCGCTATCCTGCCAACTCTATCGTGGTCG
GGGCGACTACGCGTGCGGCCCGCACGGCTA
GCCCACCTGTGACGAGGTAGGGGTAGGGCG
CCCAGGGTCTACAACATCCACAGCCGGACC
CCACGATAGAGTTGGCAGGATAGCGGGTGA
TTGGCAGGATAGCGGGTGACGGTCCGGCTG
CAGGGATACCCACCCACCATCCCAGGGTCT
CCTGGTGTTCCCTTTAAGCGAAGGTGGCTC
AGCGGCCGCCCGCCTACAACCTGGAGGCCG
ACAGGGATACCCACCCACCATCCCAGGGTC
TAAGCGAAGGTGGCTCCACAGTTGGGGCAT
CCAAAGGGGAAGAGGATGATGGCCAGGAAG
TGGGATGGTGGGTGGGTATCCCTGTGGGCG
CAGCAAATGAACCCAAAGGGGAAGAGGATG
TACGGGCCCACCTGTGACGAGGTAGGGGTA
CTACGCGTGCGGCCCGCACGGCTACGGCGC
GGCGGGCGGCCGCTCCTGCAGCAGCGGCTT
GACCACAAGCCGCTGCTGCAGGAGCGGCCG
CTGCCCGTCTCTGCTGCAGGGTTGGGGTGC
CCCACCTGTGACGAGGTAGGGGTAGGGCGG
TGATGGCCAGGAAGATGCCCAGGAAGGTGA
GGGCCTGGTGTTCCCTTTAAGCGAAGGTGG
CTGTGGATGTTGTAGACCCTGGGATGGTGG
AGGCAAAACAGCAAATGAACCCAAAGGGGA
GCCGCCCTACCCCTACCTCGTCACAGGTGG
GCAAAACAGCAAATGAACCCAAAGGGGAAG
GTCGTAGGAGGCTGTCCTGTCTGCAGGTGA
GGCCGGCCAGGGCGACTACGCGTGCGGCCC
GGCCGCTCCTGCAGCAGCGGCTTGTGGTCC
GGCAAAACAGCAAATGAACCCAAAGGGGAA
CGCCTACAACCTGGAGGCCGGCCAGGGCGA
TGCGGGCCGCACGCGTAGTCGCCCTGGCCG
CCTGCAGCAGCGGCTTGTGGTCCATGGCGG
TCTGCTGCAGGGTTGGGGTGCTGGAGGACT
GCCGGCCTCCAGGTTGTAGGCGGGCGGCCG
CCGGCTGTGGATGTTGTAGACCCTGGGATG
CCATGGACCACAAGCCGCTGCTGCAGGAGC
TGTGACGAGGTAGGGGTAGGGCGGCGGCGG
TGGATGTTGTAGACCCTGGGATGGTGGGTG
AGGAGCGGCCGCCCGCCTACAACCTGGAGG
CTGGCCGGCCTCCAGGTTGTAGGCGGGCGG
GGATGTTGTAGACCCTGGGATGGTGGGTGG
ACGAGGTAGGGGTAGGGCGGCGGCGGGGGC
AGGATGATGGCCAGGAAGATGCCCAGGAAG
CTCTGCCCGTCTCTGCTGCAGGGTTGGGGT
GACGAGGTAGGGGTAGGGCGGCGGCGGGGG
GTTGTAGACCCTGGGATGGTGGGTGGGTAT
GGCGGGGATGGCGCCGTAGCCGTGCGGGCC
GGGTTCATTTGCTGTTTTGCCTTGAGGAAG
CGAGGTAGGGGTAGGGCGGCGGCGGGGGCG
CCTGGCCGGCCTCCAGGTTGTAGGCGGGCG
CTGGCCATCATCCTCTTCCCCTTTGGGTTC
TGAACCCAAAGGGGAAGAGGATGATGGCCA
GAGGTAGGGGTAGGGCGGCGGCGGGGGCGC
GCCGCCCGCCTACAACCTGGAGGCCGGCCA
GGCCGCACGCGTAGTCGCCCTGGCCGGCCT
TGTTGTAGACCCTGGGATGGTGGGTGGGTA
GGATAGCGGGTGACGGTCCGGCTGTGGATG
AACTGTGGAGCCACCTTCGCTTAAAGGGAA
CCTGGCCATCATCCTCTTCCCCTTTGGGTT
TTAAGCGAAGGTGGCTCCACAGTTGGGGCA
TCTGCCCGTCTCTGCTGCAGGGTTGGGGTG
ACTGTGGAGCCACCTTCGCTTAAAGGGAAC
TTTAAGCGAAGGTGGCTCCACAGTTGGGGC
GCGGGGATGGCGCCGTAGCCGTGCGGGCCG
CGCCCTGGCCGGCCTCCAGGTTGTAGGCGG
TCCGGCTGTGGATGTTGTAGACCCTGGGAT
GCGTAGTCGCCCTGGCCGGCCTCCAGGTTG
CCGCCTACAACCTGGAGGCCGGCCAGGGCG
GCTGGAGGACTGCTTCACCTTCCTGGGCAT
GCTTCACCTTCCTGGGCATCTTCCTGGCCA
GCGGCGGCGGGGGCGCGGCGGGGATGGCGC
GTCTCTGCTGCAGGGTTGGGGTGCTGGAGG
TAGGGCGGCGGCGGGGGCGCGGCGGGGATG
TGCTGGAGGACTGCTTCACCTTCCTGGGCA
GGTAGGGCGGCGGCGGGGGCGCGGCGGGGA
AGGGGTAGGGCGGCGGCGGGGGCGCGGCGG
GTAGGGCGGCGGCGGGGGCGCGGCGGGGAT;

or, a sequence which possesses at least 80, 85, 90, 95 or 95-99% sequence identity to any of the foregoing sequences; or
(v) any anti-BRI3 agent utilizing CRISPR/Cas9 with an inactivated endonuclease, referred to as “dead”Cas9 or “dCas9.”

It is another specific object of the invention to provide novel methods of treatment or prevention as above, wherein the active agent that modifies the expression and/or function comprises a clustered Regularly Interspaced Short Palindromic Repeat (CRISPR)/Cas gene editing agent, wherein the active agent comprises or consists of one or more short-guide RNAs having sequences selected from one or more of oligonucleotide sequences AACTCTATCGTGGTCGTAGG, CGTCACAGGTGGGCCCGTAA, GACTACGCGTGCGGCCCGCA (depicted here in “sense” orientation) and optionally inactivated endonuclease, further optionally “dead”Cas9 or “dCas9.”

It is another specific object of the invention to provide novel methods of treatment or prevention as above, further comprising administering at least one other active agent used to treat or prevent the neurodegenerative disease or neuroinflammation, optionally Parkinson's Diseases, further optionally wherein the at least one other active agent is levodopa, carbidopa, a dopamine agonist (e.g., pramipexole, ropinirole, rotigotine, apomorphine), a monoamine oxidase B (MAO B) inhibitor (e.g., selegiline, rasagiline, safinamide), a catechol O-methyltrasnferase (COMT) inhibitor (e.g., entacapone, tolcapone), an anticholinergic (e.g., benztropine), or amantadine, or any combination thereof, further optionally comprising administering deep brain stimulation (DBS). It is another specific object of the invention to provide novel methods of treatment or prevention as above, further comprising any one or more of the following:
(i) increasing CD16⁺ monocytes;
(ii) increasing B cells;
(iii) increasing dendritic cells;
(iv) reducing CD14⁺ monocytes; and
(v) reducing CD4⁺ T cells,
   wherein optionally wherein the increasing and/or reducing in any one or more of (i)-(v) at least takes place in PBMCs.

It is another specific object of the invention to provide novel methods of treatment or prevention as above, further comprising detecting the expression and/or function of one or more of BRI3, FAM89B, PCBP1, ATP5F1E (or ATP5E), SH2B2, LMO2, TAF10, MAP2K2, TLE4, GRK6, RNF187, TNNT1, PTP4A2, SIAH2, YBX3, LAMP1, RPL8, EID2, CAPG, SRM, TMSB10, FYB, HLA-E, GLRX5, SEC61G, TMEM9, DBP, XRRA1, BLOC1S4, TUFM, HDDC2, EVL, UBB, RAC2, OAS1, LSP1, or ARF5, the expression of which is to be increased or decreased, wherein said detecting occurs prior, during and/or after said administrating, optionally wherein the detecting comprises detecting in one or more samples from the treated subject, optionally a blood sample and/or a brain sample.

It is another specific object of the invention to provide methods of determining whether a subject has a neurodegenerative disease associated with neuroinflammation or whether a subject is at increased risk of developing a neurodegenerative disease associated with neuroinflammation, comprising:
(a) measuring the expression of BRI3, FAM89B, PCBP1, ATP5F1E (or ATP5E), SH2B2, LMO2, TAF10, MAP2K2, TLE4, GRK6, RNF187, TNNT1, PTP4A2, SIAH2, YBX3, LAMP1, RPL8, EID2, CAPG, SRM, TMSB10, FYB, HLA-E, GLRX5, SEC61G, TMEM9, DBP, XRRA1, BLOC1S4, TUFM, HDDC2, EVL, UBB, RAC2, OAS1, LSP1, or ARF5, or any combination thereof in the subject or in a sample from the subject, and optionally measuring the quantity (number and/or percentage) of CD16⁺ monocytes, B cells, dendritic cells, CD14⁺ monocytes, and CD4⁺ T cells in the sample; and
(b) determining that the subject has a neurodegenerative disease if:
(i) the expression of BRI3, FAM89B, PCBP1, ATP5F1E (or ATP5E), SH2B2, LMO2, TAF10, MAP2K2, TLE4, GRK6, RNF187, TNNT1, PTP4A2, SIAH2, YBX3, LAMP1, RPL8, EID2, CAPG, SRM, TMSB10, FYB, HLA-E, GLRX5, SEC61G, TMEM9, DBP, XRRA1, BLOC1S4, or TUFM, or any combination thereof is higher than a normal control; and/or
(ii) the expression of HDDC2, EVL, UBB, RAC2, OAS1, LSP1, or ARF5, or any combination thereof is lower than a normal control,
and optionally if:
(iii) the quantity of CD16⁺ monocytes, B cells, and/or dendritic cells is reduced; and/or
(iv) the quantity of CD14⁺ monocytes and/or CD4⁺ T cells is increased, optionally wherein the neurodegenerative disease a neurodegenerative disease associated with neuroinflammation is PD.

It is another specific object of the invention to provide a method of determining whether a subject has inflammation associated with a neurodegenerative disease or is at risk of developing inflammation associated with a neurodegenerative disease, comprising:
(a) measuring the expression of BRI3, FAM89B, PCBP1, ATP5F1E (or ATP5E), SH2B2, LMO2, TAF10, MAP2K2, TLE4, GRK6, RNF187, TNNT1, PTP4A2, SIAH2, YBX3, LAMP1, RPL8, EID2, CAPG, SRM, TMSB10, FYB, HLA-E, GLRX5, SEC61G, TMEM9, DBP, XRRA1, BLOC1S4, TUFM, HDDC2, EVL, UBB, RAC2, OAS1, LSP1, or ARF5, or any combination thereof in the subject or in a sample from the subject, and optionally measuring the quantity (number and/or percentage) of CD16⁺ monocytes, B cells, dendritic cells, CD14⁺ monocytes, and CD4⁺ T cells in the sample; and
(b) determining that the subject has inflammation associated with a neurodegenerative disease if:
(i) the expression of BRI3, FAM89B, PCBP1, ATP5F1E (or ATP5E), SH2B2, LMO2, TAF10, MAP2K2, TLE4, GRK6, RNF187, TNNT1, PTP4A2, SIAH2, YBX3, LAMP1, RPL8, EID2, CAPG, SRM, TMSB10, FYB, HLA-E, GLRX5, SEC61G, TMEM9, DBP, XRRA1, BLOC1S4, or TUFM, or any combination thereof is higher than a control; and/or
(ii) the expression of HDDC2, EVL, UBB, RAC2, OAS1, LSP1, or ARF5, or any combination thereof is lower than a control,
and optionally if:
(iii) the quantity of CD16⁺ monocytes, B cells, and/or dendritic cells is reduced; and/or
(iv) the quantity of CD14⁺ monocytes and/or CD4⁺ T cells is increased, optionally wherein the neurodegenerative disease is PD.

It is another specific object of the invention to provide methods of determining whether a subject has a neurodegenerative disease associated with neuroinflammation or whether a subject is at increased risk of developing a neurodegenerative disease associated with neuroinflammation as above, further comprising one or more of the following:
(I) in (a), at least the expression of BRI3 is measured;
(II) in (b), at least the expression of BRI3 is higher than the control;
(III) in (b) (i), the expression is higher than the control by about 25%, about 30%, about 40%, about 45%, about 50%, about 55% about 60%, about 65%, about 70%, about 75%, about 80%, about 85%, about 90%, about 95%, about 100%, about 150%, about 200%, about 300%, about 400%, about 500%, about 600%, about 800%, about 900%, about 1000%, about 2000%, about 3000%, about 4000%, about 5000%, or about 10000%; and/or
(IV) in (b) (ii), the expression is lower than the control by about 25%, about 30%, about 40%, about 45%, about 50%, about 55% about 60%, about 65%, about 70%, about 75%, about 80%, about 85%, about 90%, about 95%, or about 100%.
method of determining whether the severity and/or stage of and/or inflammation associated with a neurodegenerative disease has increased in a subject, comprising:
(a) measuring the expression of BRI3, FAM89B, PCBP1, ATP5F1E (or ATP5E), SH2B2, LMO2, TAF10, MAP2K2, TLE4, GRK6, RNF187, TNNT1, PTP4A2, SIAH2, YBX3, LAMP1, RPL8, EID2, CAPG, SRM, TMSB10, FYB, HLA-E, GLRX5, SEC61G, TMEM9, DBP, XRRA1, BLOC1S4, TUFM, HDDC2, EVL, UBB, RAC2, OAS1, LSP1, or ARF5, or any combination thereof in the subject or in a sample from the subject at a first time point and a second time point, and optionally measuring the quantity (number and/or percentage) of CD16⁺ monocytes, B cells, dendritic cells, CD14⁺ monocytes, and CD4+ T cells in the subject or in the sample at the first time point and the second time point; and
(b) determining that the severity and/or stage and/or inflammation has increased if:
(i) the expression of BRI3, FAM89B, PCBP1, ATP5F1E (or ATP5E), SH2B2, LMO2, TAF10, MAP2K2, TLE4, GRK6, RNF187, TNNT1, PTP4A2, SIAH2, YBX3, LAMP1, RPL8, EID2, CAPG, SRM, TMSB10, FYB, HLA-E, GLRX5, SEC61G, TMEM9, DBP, XRRA1, BLOC1S4, or TUFM, or any combination thereof is higher at the second time point than the first time point; and/or
(ii) the expression of HDDC2, EVL, UBB, RAC2, OAS1, LSP1, or ARF5, or any combination thereof is lower at the second time point than the first time point,
and optionally if:
(iii) the quantity of CD16⁺ monocytes, B cells, and/or dendritic cells is lower at the second time point than the first time point; and/or
(iv) the quantity of CD14⁺ monocytes and/or CD4⁺ T cells is higher at the second time point than the first time point,
optionally wherein the neurodegenerative disease is PD,
further optionally wherein the method comprises one or more of the following:
(I) in (a), at least the expression of BRI3 is measured;
(II) in (b), at least the expression of BRI3 is higher at the second time point than the first time point;
(III) in (b) (i), the expression is higher at the second time point than the first time point by about 25%, about 30%, about 40%, about 45%, about 50%, about 55% about 60%, about 65%, about 70%, about 75%, about 80%, about 85%, about 90%, about 95%, about 100%, about 150%, about 200%, about 300%, about 400%, about 500%, about 600%, about 800%, about 900%, about 1000%, about 2000%, about 3000%, about 4000%, about 5000%, or about 10000%;
(IV) in (b) (ii), the expression is lower at the second time point than the first time point by about 25%, about 30%, about 40%, about 45%, about 50%, about 55% about 60%, about 65%, about 70%, about 75%, about 80%, about 85%, about 90%, about 95%, or about 100%;
(V) in (b) (iii), the quantity is lower at the second time point than the first time point by about 25%, about 30%, about 40%, about 45%, about 50%, about 55% about 60%, about 65%, about 70%, about 75%, about 80%, about 85%, about 90%, about 95%, or about 100%; and/or
(VI) in (b) (iv), the quantity is higher at the second time point than the first time point by about 25%, about 30%, about 40%, about 45%, about 50%, about 55% about 60%, about 65%, about 70%, about 75%, about 80%, about 85%, about 90%, about 95%, about 100%, about 150%, about 200%, about 300%, about 400%, about 500%, about 600%, about 800%, about 900%, about 1000%, about 2000%, about 3000%, about 4000%, about 5000%, or about 10000%.

It is another specific object of the invention to provide novel methods of determining whether a therapy or prophylaxis for a neurodegenerative disease is effective in a subject, comprising:
(a) measuring the expression of BRI3, FAM89B, PCBP1, ATP5F1E (or ATP5E), SH2B2, LMO2, TAF10, MAP2K2, TLE4, GRK6, RNF187, TNNT1, PTP4A2, SIAH2, YBX3, LAMP1, RPL8, EID2, CAPG, SRM, TMSB10, FYB, HLA-E, GLRX5, SEC61G, TMEM9, DBP, XRRA1, BLOC1S4, TUFM, HDDC2, EVL, UBB, RAC2, OAS1, LSP1, or ARF5, or any combination thereof in the subject or in a sample from the subject before and at one or more time points after starting the therapy, and optionally measuring the quantity (number and/or percentage) of CD16⁺ monocytes, B cells, dendritic cells, CD14⁺ monocytes, and CD4⁺ T cells in the subject or in the sample before and at one or more time points after starting the therapy; and
(b) determining that the therapy is effective if:
(i) the expression of BRI3, FAM89B, PCBP1, ATP5F1E (or ATP5E), SH2B2, LMO2, TAF10, MAP2K2, TLE4, GRK6, RNF187, TNNT1, PTP4A2, SIAH2, YBX3, LAMP1, RPL8, EID2, CAPG, SRM, TMSB10, FYB, HLA-E, GLRX5, SEC61G, TMEM9, DBP, XRRA1, BLOC1S4, or TUFM, or any combination thereof is lower at least one time point after starting the therapy compared to before starting the therapy; and/or
(ii) the expression of HDDC2, EVL, UBB, RAC2, OAS1, LSP1, or ARF5, or any combination thereof is higher at least one time point after starting the therapy compared to before starting the therapy,
and optionally if:
(iii) the quantity of CD16⁺ monocytes, B cells, and/or dendritic cells is lower at least one time point after starting the therapy compared to before starting the therapy; and/or
(iv) the quantity of CD14⁺ monocytes and/or CD4⁺ T cells is higher least one time point after starting the therapy compared to before starting the therapy, optionally wherein the neurodegenerative disease is PD, further optionally wherein the method comprises one or more of the following:
(I) in (a), at least the expression of BRI3 is measured;
(II) in (b), at least the expression of BRI3 is lower at least one time point after starting the therapy compared to before starting the therapy;
(III) in (b) (i), the expression is lower at least one time point after starting the therapy compared to before starting the therapy by about 25%, about 30%, about 40%, about 45%, about 50%, about 55% about 60%, about 65%, about 70%, about 75%, about 80%, about 85%, about 90%, about 95%, or about 100%;
(IV) in (b) (ii), the expression is higher at least one time point after starting the therapy compared to before starting the therapy by about 25%, about 30%, about 40%, about 45%, about 50%, about 55% about 60%, about 65%, about 70%, about 75%, about 80%, about 85%, about 90%, about 95%, about 100%, about 150%, about 200%, about 300%, about 400%, about 500%, about 600%, about 800%, about 900%, about 1000%, about 2000%, about 3000%, about 4000%, about 5000%, or about 10000%;
(V) in (b) (iii), the quantity is higher at least one time point after starting the therapy compared to before starting the therapy by about 25%, about 30%, about 40%, about 45%, about 50%, about 55% about 60%, about 65%, about 70%, about 75%, about 80%, about 85%, about 90%, about 95%, about 100%, about 150%, about 200%, about 300%, about 400%, about 500%, about 600%, about 800%, about 900%, about 1000%, about 2000%, about 3000%, about 4000%, about 5000%, or about 10000%; and/or
(VI) in (b) (iv), the quantity is lower at least one time point after starting the therapy compared to before starting the therapy by about 25%, about 30%, about 40%, about 45%, about 50%, about 55% about 60%, about 65%, about 70%, about 75%, about 80%, about 85%, about 90%, about 95%, or about 100%.

It is another specific object of the invention to provide novel methods of detection as above wherein, in (a), measuring is via one or more of RNA sequencing, RNA-sequencing at single cell resolution, DNA array, flow cytometry, histochemistry, protein detection (optionally by use of bead-based or solid phase protein detection methods, further optionally wherein protein detection is effected by the use of one or more of an immunosorbent assay, gel electrophoresis, SDS-PAGE (polyacrylamide gel electrophoresis), Liquid chromatography-mass spectrometry (LC-MS), HPLC, ELISA, immunoelectrophoresis, immunostaining, Western blot, protein colorimetric assay, flow cytometry, electron microscopy, an enzyme assay, immune fluorescence, spectrophotometry, and the like) or imaging, optionally wherein the sample comprises PBMCs, monocytes, dendritic cells, and/or central nervous system (CNS) cells, optionally wherein the monocytes are circulating monocytes, further optionally wherein the monocytes are CD16⁺ monocytes, CD14⁺ monocytes, and/or meningeal monocytes, and yet further optionally wherein the CNS cells comprise microglia.

It is another specific object of the invention to provide novel methods of screening for a therapeutic agent for a neurodegenerative disease, comprising:
(a) applying a candidate therapeutic agent to (1) one or more cells derived from a subject with the neurodegenerative disease, (2) one or more neurodegenerative disease cell line cells, or (3) a cell or tissue culture comprising a sample derived from a neurodegenerative disease patient;
(b) after step (a), measuring the expression of BRI3, FAM89B, PCBP1, ATP5F1E (or ATP5E), SH2B2, LMO2, TAF10, MAP2K2, TLE4, GRK6, RNF187, TNNT1, PTP4A2, SIAH2, YBX3, LAMP1, RPL8, EID2, CAPG, SRM, TMSB10, FYB, HLA-E, GLRX5, SEC61G, TMEM9, DBP, XRRA1, BLOC1S4, TUFM, HDDC2, EVL, UBB, RAC2, OAS1, LSP1, or ARF5, or any combination thereof in said (1), (2), or (3), and optionally measuring the quantity (number and/or percentage) of CD16⁺ monocytes, B cells, dendritic cells, CD14⁺ monocytes, and CD4⁺ T cells in said (1), (2), or (3); and
(c) determining that the candidate therapeutic agent is effective if:
(i) the expression of BRI3, FAM89B, PCBP1, ATP5F1E (or ATP5E), SH2B2, LMO2, TAF10, MAP2K2, TLE4, GRK6, RNF187, TNNT1, PTP4A2, SIAH2, YBX3, LAMP1, RPL8, EID2, CAPG, SRM, TMSB10, FYB, HLA-E, GLRX5, SEC61G, TMEM9, DBP, XRRA1, BLOC1S4, or TUFM, or any combination thereof is downregulated compared to an untreated or placebo control; and/or
(ii) the expression of HDDC2, EVL, UBB, RAC2, OAS1, LSP1, or ARF5, or any combination thereof is upregulated compared to an untreated or placebo control,
and optionally if:
(iii) the quantity of CD16⁺ monocytes, B cells, and/or dendritic cells is lower compared to an untreated or placebo control; and/or
(iv) the quantity of CD14⁺ monocytes and/or CD4⁺ T cells is higher compared to an untreated or placebo control,
optionally wherein the neurodegenerative disease is PD,
further optionally wherein the method comprises one or more of the following:
(I) in (a), at least the expression of BRI3 is measured;
(II) in (b), at least the expression of BRI3 is downregulated compared to an untreated or placebo control;
(III) in (b) (i), the expression is downregulated compared to an untreated or placebo control by about 25%, about 30%, about 40%, about 45%, about 50%, about 55% about 60%, about 65%, about 70%, about 75%, about 80%, about 85%, about 90%, about 95%, or about 100%;
(IV) in (b) (ii), the expression is upregulated compared to an untreated or placebo control by about 25%, about 30%, about 40%, about 45%, about 50%, about 55% about 60%, about 65%, about 70%, about 75%, about 80%, about 85%, about 90%, about 95%, about 100%, about 150%, about 200%, about 300%, about 400%, about 500%, about 600%, about 800%, about 900%, about 1000%, about 2000%, about 3000%, about 4000%, about 5000%, or about 10000%;
(V) in (b) (iii), the quantity is higher compared to an untreated or placebo control by about 25%, about 30%, about 40%, about 45%, about 50%, about 55% about 60%, about 65%, about 70%, about 75%, about 80%, about 85%, about 90%, about 95%, about 100%, about 150%, about 200%, about 300%, about 400%, about 500%, about 600%, about 800%, about 900%, about 1000%, about 2000%, about 3000%, about 4000%, about 5000%, or about 10000%; and/or
(VI) in (b) (iv), the quantity is lower compared to an untreated or placebo control by about 25%, about 30%, about 40%, about 45%, about 50%, about 55% about 60%, about 65%, about 70%, about 75%, about 80%, about 85%, about 90%, about 95%, or about 100%.

It is another specific object of the invention to provide novel methods of detection or screening as above, wherein:
(A) the one or more cells in (1) or (2) comprise a PBMC, a blood cell, an immune cell, a monocyte, a dendritic cell, and/or a central nervous system (CNS) cell, optionally wherein the monocyte is a circulating monocyte, further optionally wherein the monocyte is a CD16⁺ monocyte, a CD14⁺ monocyte, and/or a meningeal monocyte, and yet further optionally wherein the CNS cell comprises or is a microglia; and/or
(B) the cell or tissue culture comprising a sample derived from a neurodegenerative disease patient in (3) comprises an organoid or three-dimensional cell culture.

It is another specific object of the invention to provide novel methods of predicting whether a subject with a neurodegenerative disease will respond to a therapy comprising:
(a) applying the therapy to (1) one or more cells derived from a subject with the neurodegenerative disease or (2) a cell or tissue culture comprising a sample derived from a neurodegenerative disease patient;
(b) after step (a), measuring the expression of BRI3, FAM89B, PCBP1, ATP5F1E (or ATP5E), SH2B2, LMO2, TAF10, MAP2K2, TLE4, GRK6, RNF187, TNNT1, PTP4A2, SIAH2, YBX3, LAMP1, RPL8, EID2, CAPG, SRM, TMSB10, FYB, HLA-E, GLRX5, SEC61G, TMEM9, DBP, XRRA1, BLOC1S4, TUFM, HDDC2, EVL, UBB, RAC2, OAS1, LSP1, or ARF5, or any combination thereof in said (1) or (2), and optionally measuring the quantity (number and/or percentage) of CD16⁺ monocytes, B cells, dendritic cells, CD14⁺ monocytes, and CD4⁺ T cells in said (1) or (2); and
(c) predicting that the subject will respond to the therapy if:
(i) the expression of BRI3, FAM89B, PCBP1, ATP5F1E (or ATP5E), SH2B2, LMO2, TAF10, MAP2K2, TLE4, GRK6, RNF187, TNNT1, PTP4A2, SIAH2, YBX3, LAMP1, RPL8, EID2, CAPG, SRM, TMSB10, FYB, HLA-E, GLRX5, SEC61G, TMEM9, DBP, XRRA1, BLOC1S4, or TUFM, or any combination thereof is downregulated compared to an untreated or placebo control; and/or
(ii) the expression of HDDC2, EVL, UBB, RAC2, OAS1, LSP1, or ARF5, or any combination thereof is upregulated compared to an untreated or placebo control,
and optionally if:
(iii) the quantity of CD16⁺ monocytes, B cells, and/or dendritic cells is lower compared to an untreated or placebo control; and/or
(iv) the quantity of CD14⁺ monocytes and/or CD4⁺ T cells is higher compared to an untreated or placebo control,
optionally wherein the neurodegenerative disease is PD,
further optionally wherein the method comprises one or more of the following:
(I) in (a), at least the expression of BRI3 is measured;
(II) in (b), at least the expression of BRI3 is downregulated compared to an untreated or placebo control;
(III) in (b) (i), the expression is downregulated compared to an untreated or placebo control by about 25%, about 30%, about 40%, about 45%, about 50%, about 55% about 60%, about 65%, about 70%, about 75%, about 80%, about 85%, about 90%, about 95%, or about 100%;
(IV) in (b) (ii), the expression is upregulated compared to an untreated or placebo control by about 25%, about 30%, about 40%, about 45%, about 50%, about 55% about 60%, about 65%, about 70%, about 75%, about 80%, about 85%, about 90%, about 95%, about 100%, about 150%, about 200%, about 300%, about 400%, about 500%, about 600%, about 800%, about 900%, about 1000%, about 2000%, about 3000%, about 4000%, about 5000%, or about 10000%;
(V) in (b) (iii), the quantity is higher compared to an untreated or placebo control by about 25%, about 30%, about 40%, about 45%, about 50%, about 55% about 60%, about 65%, about 70%, about 75%, about 80%, about 85%, about 90%, about 95%, about 100%, about 150%, about 200%, about 300%, about 400%, about 500%, about 600%, about 800%, about 900%, about 1000%, about 2000%, about 3000%, about 4000%, about 5000%, or about 10000%; and/or
(VI) in (b) (iv), the quantity is lower compared to an untreated or placebo control by about 25%, about 30%, about 40%, about 45%, about 50%, about 55% about 60%, about 65%, about 70%, about 75%, about 80%, about 85%, about 90%, about 95%, or about 100%.

It is another specific object of the invention to provide novel methods as above, wherein:
(A) the one or more cells in (1) comprise a PBMC, a blood cell, an immune cell, a monocyte, a dendritic cell, and/or a central nervous system (CNS) cell, optionally wherein the monocyte is a circulating monocyte, further optionally wherein the monocyte is a CD16⁺ monocyte, a CD14⁺ monocyte, and/or meningeal monocyte, and yet further optionally wherein the CNS cell comprises or is a microglia; and/or
(B) the cell or tissue culture comprising a sample derived from a neurodegenerative disease patient in (2) comprises an organoid or three-dimensional cell culture.

It is another specific object of the invention to provide novel screening methods of as above, further comprising:
(d) if in step (c) the subject is predicted to respond to the therapy, administering said therapy to the subject.

It is another specific object of the invention to provide novel treatment, prevention, detection, diagnostic or screening methods as above, wherein the neurodegenerative disease includes any of Parkinson's disease (PD), Alzheimer's disease (AD), multiple sclerosis (MS), Lewy body disease or Lewy body dementia (LBD), multiple system atrophy (MSA), progressive supranuclear palsy (PSP), amyotrophic lateral sclerosis (ALS) or motor neurone diseases (MND), Huntington's Disease (HD), spinocerebellar ataxia (SCA), Friedreich's ataxia (FA), spinal muscular atrophy (SMA), or prion disease (e.g., Creutzfeldt-Jakob disease (CJD)), optionally wherein the neurodegenerative diseases is PD.

It is another specific object of the invention to provide an agent for treating or preventing or slowing the onset of a neurodegenerative disease, selected from:
(I) one which modifies the expression or function of BRI3, FAM89B, PCBP1, ATP5F1E (or ATP5E), SH2B2, LMO2, TAF10, MAP2K2, TLE4, GRK6, RNF187, TNNT1, PTP4A2, SIAH2, YBX3, LAMP1, RPL8, EID2, CAPG, SRM, TMSB10, FYB, HLA-E, GLRX5, SEC61G, TMEM9, DBP, XRRA1, BLOC1S4, TUFM, HDDC2, EVL, UBB, RAC2, OAS1, LSP1, or ARF5, or any combination thereof;
(II) one which decreases the expression of BRI3, FAM89B, PCBP1, ATP5F1E (or ATP5E), SH2B2, LMO2, TAF10, MAP2K2, TLE4, GRK6, RNF187, TNNT1, PTP4A2, SIAH2, YBX3, LAMP1, RPL8, EID2, CAPG, SRM, TMSB10, FYB, HLA-E, GLRX5, SEC61G, TMEM9, DBP, XRRA1, BLOC1S4, or TUFM, or any combination thereof;
(III) one which suppresses, blocks, or inhibits the function of BRI3, FAM89B, PCBP1, ATP5F1E (or ATP5E), SH2B2, LMO2, TAF10, MAP2K2, TLE4, GRK6, RNF187, TNNT1, PTP4A2, SIAH2, YBX3, LAMP1, RPL8, EID2, CAPG, SRM, TMSB10, FYB, HLA-E, GLRX5, SEC61G, TMEM9, DBP, XRRA1, BLOC1S4, or TUFM, or any combination thereof;
(IV) one which decreases the expression or function of BRI3;
(V) one which increases the expression of HDDC2, EVL, UBB, RAC2, OAS1, LSP1, or ARF5, or any combination thereof;
(VI) one which enhances the function of HDDC2, EVL, UBB, RAC2, OAS1, LSP1, or ARF5, or any combination thereof; or
(VII) any combination of (I)-(VI)
optionally wherein the neurodegenerative disease is PD.

It is another specific object of the invention to provide an agent for treating or preventing or slowing the onset of a neurodegenerative disease, selected from a clustered Regularly Interspaced Short Palindromic Repeat (CRISPR)/Cas gene editing agent, a zinc-finger nuclease (ZFN) gene editing agent, a transcription activator-like effector nuclease (TALEN) gene editing agent, a transposase-based gene therapy, an siRNA, an shRNA, an miRNA, an aptamer, an antibody, an antigen-binding antibody fragment (e.g., scFv, Fab, Fab′, (Fab′)2), a chimeric antigen receptor (CAR)-expressing cell, a peptide, a small molecule, a polymer, an expression vector encoding a gene of interest, or any combination thereof.

It is another specific object of the invention to provide an agent for treating or preventing or slowing the onset of a neurodegenerative disease, as above wherein the active agent comprises or consists of one or more of the following:
(i) a CRISPR/Cas gene editing agent against BRI3;
(ii) a short-guide RNA (sgRNA) selected from the oligonucleotide sequences AACTCTATCGTGGTCGTAGG, CGTCACAGGTGGGCCCGTAA, GACTACGCGTGCGGCCCGCA (depicted here in “sense” orientation),
(iii) a sgRNA targeting BRI3 falling within or including any of the following genomic sequences: GAGGAAGCGACGATGCCCCAACTGTGGAGC, ACCACGATAGAGTTGGCAGGATAGCGGGTG, AGTTGGGGCATCGTCGCTTCCTCAAGGCAA, TTACGGGCCCACCTGTGACGAGGTAGGGGT, GCCCTACCCCTACCTCGTCACAGGTGGGCC, TCCTGCCAACTCTATCGTGGTCGTAGGAGG, CCCGCTATCCTGCCAACTCTATCGTGGTCG, GGGCGACTACGCGTGCGGCCCGCACGGCTA, GCCCACCTGTGACGAGGTAGGGGTAGGGCG, CCCAGGGTCTACAACATCCACAGCCGGACC, CCACGATAGAGTTGGCAGGATAGCGGGTGA, TTGGCAGGATAGCGGGTGACGGTCCGGCTG, CAGGGATACCCACCCACCATCCCAGGGTCT, CCTGGTGTTCCCTTTAAGCGAAGGTGGCTC (as annotated in Gene ID 25798, NM_015379.5, or identical BRI3 sequences in prior or future annotations),
(iv) a sgRNA sequence selected from one comprising or consisting of any of the following nucleic acid sequences:

GAGGAAGCGACGATGCCCCAACTGTGGAGC
GAGGAAGCGACGATGCCCCAACTGTGGAGC
GAGGAAGCGACGATGCCCCAACTGTGGAGC
GAGGAAGCGACGATGCCCCAACTGTGGAGC
GAGGAAGCGACGATGCCCCAACTGTGGAGC
GAGGAAGCGACGATGCCCCAACTGTGGAGC
GAGGAAGCGACGATGCCCCAACTGTGGAGC
GAGGAAGCGACGATGCCCCAACTGTGGAGC
GAGGAAGCGACGATGCCCCAACTGTGGAGC
GAGGAAGCGACGATGCCCCAACTGTGGAGC
GAGGAAGCGACGATGCCCCAACTGTGGAGC
ACCACGATAGAGTTGGCAGGATAGCGGGTG
AGTTGGGGCATCGTCGCTTCCTCAAGGCAA
TTACGGGCCCACCTGTGACGAGGTAGGGGT
GCCCTACCCCTACCTCGTCACAGGTGGGCC
TCCTGCCAACTCTATCGTGGTCGTAGGAGG
CCCGCTATCCTGCCAACTCTATCGTGGTCG
GGGCGACTACGCGTGCGGCCCGCACGGCTA
GCCCACCTGTGACGAGGTAGGGGTAGGGCG
CCCAGGGTCTACAACATCCACAGCCGGACC
CCACGATAGAGTTGGCAGGATAGCGGGTGA
TTGGCAGGATAGCGGGTGACGGTCCGGCTG
CAGGGATACCCACCCACCATCCCAGGGTCT
CCTGGTGTTCCCTTTAAGCGAAGGTGGCTC
AGCGGCCGCCCGCCTACAACCTGGAGGCCG
ACAGGGATACCCACCCACCATCCCAGGGTC
TAAGCGAAGGTGGCTCCACAGTTGGGGCAT
CCAAAGGGGAAGAGGATGATGGCCAGGAAG
TGGGATGGTGGGTGGGTATCCCTGTGGGCG
CAGCAAATGAACCCAAAGGGGAAGAGGATG
TACGGGCCCACCTGTGACGAGGTAGGGGTA
CTACGCGTGCGGCCCGCACGGCTACGGCGC
GGCGGGCGGCCGCTCCTGCAGCAGCGGCTT
GACCACAAGCCGCTGCTGCAGGAGCGGCCG
CTGCCCGTCTCTGCTGCAGGGTTGGGGTGC
CCCACCTGTGACGAGGTAGGGGTAGGGCGG
TGATGGCCAGGAAGATGCCCAGGAAGGTGA
GGGCCTGGTGTTCCCTTTAAGCGAAGGTGG
CTGTGGATGTTGTAGACCCTGGGATGGTGG
AGGCAAAACAGCAAATGAACCCAAAGGGGA
GCCGCCCTACCCCTACCTCGTCACAGGTGG
GCAAAACAGCAAATGAACCCAAAGGGGAAG
GTCGTAGGAGGCTGTCCTGTCTGCAGGTGA
GGCCGGCCAGGGCGACTACGCGTGCGGCCC
GGCCGCTCCTGCAGCAGCGGCTTGTGGTCC
GGCAAAACAGCAAATGAACCCAAAGGGGAA
CGCCTACAACCTGGAGGCCGGCCAGGGCGA
TGCGGGCCGCACGCGTAGTCGCCCTGGCCG
CCTGCAGCAGCGGCTTGTGGTCCATGGCGG
TCTGCTGCAGGGTTGGGGTGCTGGAGGACT
GCCGGCCTCCAGGTTGTAGGCGGGCGGCCG
CCGGCTGTGGATGTTGTAGACCCTGGGATG
CCATGGACCACAAGCCGCTGCTGCAGGAGC
TGTGACGAGGTAGGGGTAGGGCGGCGGCGG
TGGATGTTGTAGACCCTGGGATGGTGGGTG
AGGAGCGGCCGCCCGCCTACAACCTGGAGG
CTGGCCGGCCTCCAGGTTGTAGGCGGGCGG
GGATGTTGTAGACCCTGGGATGGTGGGTGG
ACGAGGTAGGGGTAGGGCGGCGGCGGGGGC
AGGATGATGGCCAGGAAGATGCCCAGGAAG
CTCTGCCCGTCTCTGCTGCAGGGTTGGGGT
GACGAGGTAGGGGTAGGGCGGCGGCGGGGG
GTTGTAGACCCTGGGATGGTGGGTGGGTAT
GGCGGGGATGGCGCCGTAGCCGTGCGGGCC
GGGTTCATTTGCTGTTTTGCCTTGAGGAAG
CGAGGTAGGGGTAGGGCGGCGGCGGGGGCG
CCTGGCCGGCCTCCAGGTTGTAGGCGGGCG
CTGGCCATCATCCTCTTCCCCTTTGGGTTC
TGAACCCAAAGGGGAAGAGGATGATGGCCA
GAGGTAGGGGTAGGGCGGCGGCGGGGGCGC
GCCGCCCGCCTACAACCTGGAGGCCGGCCA
GGCCGCACGCGTAGTCGCCCTGGCCGGCCT
TGTTGTAGACCCTGGGATGGTGGGTGGGTA
GGATAGCGGGTGACGGTCCGGCTGTGGATG
AACTGTGGAGCCACCTTCGCTTAAAGGGAA
CCTGGCCATCATCCTCTTCCCCTTTGGGTT
TTAAGCGAAGGTGGCTCCACAGTTGGGGCA
TCTGCCCGTCTCTGCTGCAGGGTTGGGGTG
ACTGTGGAGCCACCTTCGCTTAAAGGGAAC
TTTAAGCGAAGGTGGCTCCACAGTTGGGGC
GCGGGGATGGCGCCGTAGCCGTGCGGGCCG
CGCCCTGGCCGGCCTCCAGGTTGTAGGCGG
TCCGGCTGTGGATGTTGTAGACCCTGGGAT
GCGTAGTCGCCCTGGCCGGCCTCCAGGTTG
CCGCCTACAACCTGGAGGCCGGCCAGGGCG
GCTGGAGGACTGCTTCACCTTCCTGGGCAT
GCTTCACCTTCCTGGGCATCTTCCTGGCCA
GCGGCGGCGGGGGCGCGGCGGGGATGGCGC
GTCTCTGCTGCAGGGTTGGGGTGCTGGAGG
TAGGGCGGCGGCGGGGGCGCGGCGGGGATG
TGCTGGAGGACTGCTTCACCTTCCTGGGCA
GGTAGGGCGGCGGCGGGGGCGCGGCGGGGA
AGGGGTAGGGCGGCGGCGGGGGCGCGGCGG
GTAGGGCGGCGGCGGGGGCGCGGCGGGGAT;

or, a sequence which possesses at least 80, 85, 90, 95 or 95-99% sequence identity to any of the foregoing sequences; or
(v) any anti-BRI3 agent utilizing CRISPR/Cas9 with an inactivated endonuclease, referred to as “dead”Cas9 or “dCas9.”

It is another specific object of the invention to provide a composition for treating or preventing a neurodegenerative disease, comprising an agent as described in the previous paragraphs, optionally wherein the neurodegenerative disease is PD.

It is another specific object of the invention to provide an agent or composition for treating or preventing a neurodegenerative disease, comprising an agent as described in the previous paragraphs, wherein the neurodegenerative disease is selected from Parkinson's disease (PD), Alzheimer's disease (AD), multiple sclerosis (MS), Lewy body disease or Lewy body dementia (LBD), progressive supranuclear palsy (PSP), amyotrophic lateral sclerosis (ALS) or motor neurone diseases (MND), Huntington's Disease (HD), spinocerebellar ataxia (SCA), Friedreich's ataxia (FA), spinal muscular atrophy (SMA), or prion disease (e.g., Creutzfeldt-Jakob disease (CJD)) or is PD.

It is another specific object of the invention to provide a kit comprising:
(a) at least one primer set or at least one antibody for detecting expression of BRI3, FAM89B, PCBP1, ATP5F1E (or ATP5E), SH2B2, LMO2, TAF10, MAP2K2, TLE4, GRK6, RNF187, TNNT1, PTP4A2, SIAH2, YBX3, LAMP1, RPL8, EID2, CAPG, SRM, TMSB10, FYB, HLA-E, GLRX5, SEC61G, TMEM9, DBP, XRRA1, BLOC1S4, TUFM, HDDC2, EVL, UBB, RAC2, OAS1, LSP1, or ARF5, or any combination thereof; and
(b) an instruction sheet,
optionally wherein said at least one primer set or at least one antibody in (a) comprises a primer set or an antibody for detecting expression of BRI3.

It is another specific object of the invention to provide a kit comprising:
(a) (1) one or more cells derived from a subject with a neurodegenerative disease, (2) one or more neurodegenerative disease cell line cells, or (3) a cell or tissue culture comprising a sample derived from a neurodegenerative disease patient; and
(b) at least one primer set or at least one antibody for detecting expression of BRI3, FAM89B, PCBP1, ATP5F1E (or ATP5E), SH2B2, LMO2, TAF10, MAP2K2, TLE4, GRK6, RNF187, TNNT1, PTP4A2, SIAH2, YBX3, LAMP1, RPL8, EID2, CAPG, SRM, TMSB10, FYB, HLA-E, GLRX5, SEC61G, TMEM9, DBP, XRRA1, BLOC1S4, TUFM, HDDC2, EVL, UBB, RAC2, OAS1, LSP1, or ARF5, or any combination thereof,
optionally wherein said at least one primer set or at least one antibody in (b) comprises a primer set or an antibody for detecting expression of BRI3,
further optionally wherein the kit is for screening a therapeutic agent for treating or preventing the neurodegenerative disease,
and still further optionally wherein the neurodegenerative disease is PD.
It is another specific object of the invention to provide a kit as described above, wherein expression is detected via a chemiluminescent, fluorescent, and/or colorimetric assay.

### DETAILED DESCRIPTION OF THE INVENTION

### Definitions

Although various embodiments and examples of the present invention have been described referring to certain molecules, compositions, methods, or protocols, it is to be understood that the present invention is not limited to the particular molecules, compositions, methods, or protocols described herein, as theses may vary. It is also to be understood that the terminology used in the description is for the purpose of describing the particular versions or embodiments only and is not intended to limit the scope of the present invention which will be limited only by the appended claims.

All publications mentioned herein are incorporated herein by reference. Nothing herein is to be construed as an admission that the present invention is not entitled to antedate such disclosure by virtue of prior invention.

In the specification above and in the appended claims, all transitional phrases such as “comprising,” “including,” “having,” “containing,” “involving,” “composed of,” and the like are to be understood to be open-ended, namely, to mean including but not limited to. Only the transitional phrases “consisting of” and “consisting essentially of” shall be closed or semi-closed transitional phrases, respectively.

It must also be noted that, unless the context clearly dictates otherwise, the singular forms “a,” “an,” and “the” as used herein and in the appended claims include plural reference. Thus, the reference to “a cell” refers to one or more cells and equivalents thereof known to those skilled in the art, and so forth. Unless defined otherwise, all technical and scientific terms used herein have the same meanings as commonly understood by a person of skilled in the art.

It should be understood that, unless clearly indicated otherwise, in any methods disclosed or claimed herein that comprise more than one step, the order of the steps to be performed is not restricted by the order of the steps cited.

The term “about” or “approximately” as used herein when referring to a numerical value, such as of weight, mass, volume, concentration, or time, should not be limited to the recited numerical value but rather encompasses variations of +/−10% of a given value.

“BRI3” as used herein, also known as “brain protein I3,” “I3” or “pRGR2”, is in human encoded by the BRI3 gene in Chromosome 7 at 7q21.3 (Gene ID: 25798).

An “anti-BRI3 agent” as used herein refers to any agents that are able to target BRI3 directly or indirectly. Anti-BRI3 agents of the present invention include, but are not limited to, any single guide RNA sequence targeting BRI3 through clustered Regularly Interspaced Short Palindromic Repeat (CRISPR)/Cas gene editing agent, a zinc-finger nuclease (ZFN) gene editing agent, a transcription activator-like effector nuclease (TALEN) gene editing agent, a transposase-based gene therapy, an siRNA, an shRNA, an miRNA, an aptamer, an antibody, an antigen-binding antibody fragment (e.g., scFv, Fab, Fab′, (Fab′)2), a chimeric antigen receptor (CAR)-expressing cell, a peptide, a small molecule, a polymer, an expression vector encoding a gene of interest, or any combination thereof. In some exemplary embodiments, the anti-BRI3 agent may comprise (i) short-guide RNA including but not limited to oligonucleotide sequences AACTCTATCGTGGTCGTAGG, CGTCACAGGTGGGCCCGTAA, GACTACGCGTGCGGCCCGCA (depicted here in “sense” orientation) or may comprise (ii) CRISPR/Cas9 with an inactivated endonuclease, referred to as “dead”Cas9 or “dCas9.”

The term “antibody” or “Ab,” or “immunoglobulin” is used herein in the broadest sense and encompasses various antibody structures which specifically binds with an antigen, including but not limited to monoclonal antibodies, polyclonal antibodies, multispecific antibodies (e.g., bispecific antibodies), and/or antibody fragments (also referred to as “antigen-binding antibody fragments”). Typically, a full-size Ab (also referred to as an intact Ab) comprises two pairs of chains, each pair comprising a heavy chain (HC) and a light chain (LC). A HC typically comprises a variable region and a constant region. A LC also typically comprises a variable region and constant region. The variable region of a heavy chain (VH) typically comprises three complementarity-determining regions (CDRs), which are referred to herein as CDR 1, CDR 2, and CDR 3 (or referred to as CDR-H1, CDR-H2, CDR-H3, respectively). The constant region of a HC typically comprises a fragment crystallizable region (Fc region), which dictates the isotype of the Ab, the type of Fc receptor the Ab binds to, and therefore the effector function of the Ab. Any isotype, such as IgG1, IgG2a, IgG2b, IgG3, IgG4, IgM, IgD, IgE, IgGA1, or IgGA2, may be used. Fc receptor types include, but are not limited to, FcaR (such as FcaRI), Fca/mR, FceR (such as FceRI, FceRII), FcgR (such as FcgRI, FcgRIIA, FcgRIIB1, FcgRIIB2, FcgRIIIA, FcgRIIIB), and FcRn and their associated downstream effects are well known in the art. The variable region of a light chain (VL) also typically comprises CDRs, which are CDR 1, CDR 2, and CDR 3 (or referred to as CDR-L1, CDR-L2, CDR-L3, respectively). In some embodiments, the antigen is ACVR1C (also referred to as ALK7). Antibodies can be intact immunoglobulins derived from natural sources or from recombinant sources. A portion of an antibody that comprises a structure that enables specific binding to an antigen is referred to “antigen-binding fragment,” “AB domain,” “antigen-binding region,” or “AB region” of the Ab.

Certain amino acid modifications in the Fc region are known to modulate Ab effector functions and properties, such as, but not limited to, antibody-dependent cellular cytotoxicity (ADCC), antibody-dependent cellular phagocytosis (ADCP), complement dependent cytotoxicity (CDC), and half-life (Wang X. et al., Protein Cell. 2018 January; 9(1): 63-73; Dall'Acqua W. F. et al., J. Biol Chem. 2006 Aug. 18; 281(33):23514-24. Epub 2006 Jun 21; Monnet C. et al, Front Immunol. 2015 Feb. 4; 6:39. doi: 10.3389/fimmu.2015.00039. eCollection 2015). The mutation may be symmetrical or asymmetrical. In certain cases, antibodies with Fc regions that have asymmetrical mutation(s) (i.e., two Fc regions are not identical) may provide better functions such as ADCC (Liu Z. et al. J. Biol Chem. 2014 Feb. 7; 289(6): 3571-3590).

An IgG1-type Fc optionally may comprise one or more amino acid substitutions. Such substitutions may include, for example, N297A, N297Q, D265A, L234A, L235A, C226S, C229S, P238S, E233P, L234V, G236-deleted, P238A, A327Q, A327G, P329A, K322A, L234F, L235E, P331S, T394D, A330L, P331S, F243L, R292P, Y300L, V3051, P396L, S239D, I332E, S298A, E333A, K334A, L234Y, L235Q, G236W, S239M, H268D, D270E, K326D, A330M, K334E, G236A, K326W, S239D, E333S, S267E, H268F, 5324T, E345R, E430G, S440Y, M428L, N434S, L328F, M252Y, 5254T, T256E, and/or any combination thereof (the residue numbering is according to the EU index as in Kabat) (Dall'Acqua W. F. et al., J. Biol Chem. 2006 Aug. 18; 281(33):23514-24. Epub 2006 Jun 21; Wang X. et al., Protein Cell. 2018 January; 9(1): 63-73), or for example, N434A, Q438R, S440E, L432D, N434L, and/or any combination thereof (the residue numbering according to EU numbering). The Fc region may further comprise one or more additional amino acid substitutions. Such substitutions may include but are not limited to A330L, L234F, L235E, P3318, and/or any combination thereof (the residue numbering is according to the EU index as in Kabat). Specific exemplary substitution combinations for an IgG1-type Fc include, but not limited to: M252Y, 5254T, and T256E (“YTE” variant); M428L and N434A (“LA” variant), M428L and N434S (“LS” variant); M428L, N434A, Q438R, and S440E (“LA-RE” variant); L432D and N434L (“DEL” variant); and L234A, L235A, L432D, and N434L (“LALA-DEL” variant) (the residue numbering is according to the EU index as in Kabat). In particular embodiments, an IgG1-type Fc variant may comprise the amino acid sequence of SEQ ID NOS: 11, 12, 13, 14, 15, 16, or 17.

When the Ab is an IgG2, the Fc region optionally may comprise one or more amino acid substitutions. Such substitutions may include but are not limited to P238S, V234A, G237A, H268A, H268Q, H268E, V309L, N297A, N297Q, A330S, P331S, C232S, C233S, M252Y, 5254T, T256E, and/or any combination thereof (the residue numbering is according to the EU index as in Kabat). The Fc region optionally may further comprise one or more additional amino acid substitutions. Such substitutions may include but are not limited to M252Y, 5254T, T256E, and/or any combination thereof (the residue numbering is according to the EU index as in Kabat).

An IgG3-type Fc region optionally may comprise one or more amino acid substitutions. Such substitutions may include but are not limited to E235Y (the residue numbering is according to the EU index as in Kabat).

An IgG4-type Fc region optionally may comprise one or more amino acid substitutions. Such substitutions may include but are not limited to, E233P, F234V, L235A, G237A, E318A, S228P, L236E, S241P, L248E, T394D, M252Y, S254T, T256E, N297A, N297Q, and/or any combination thereof (the residue numbering is according to the EU index as in Kabat). The substitution may be, for example, S228P (the residue numbering is according to the EU index as in Kabat).

In some cases, the glycan of the human-like Fc region may be engineered to modify the effector function (for example, see Li T. et al., Proc Natl Acad Sci USA. 2017 Mar. 28; 114(13):3485-3490. doi: 10.1073/pnas.1702173114. Epub 2017 Mar. 13).

The term “antibody fragment” or “Ab fragment” as used herein refers to any portion or fragment of an Ab, including intact or full-length Abs that may be of any class or sub-class, including IgG and sub-classes thereof, IgM, IgE, IgA, and IgD. The term encompasses molecules constructed using one or more potions or fragments of one or more Abs. An Ab fragment can be immunoreactive portions of intact immunoglobulins. The term is used in the broadest sense and includes polyclonal and monoclonal antibodies, including intact antibodies and functional (antigen-binding) antibody fragments, including fragment antigen binding (Fab) fragments, F(ab′)2 fragments, Fab′ fragments, Fv fragments, recombinant IgG (rIgG) fragments, single chain antibody fragments, including single chain variable fragments (scFv), diabodies, and single domain antibodies (e.g., sdAb, sdFv, nanobody) fragments. The term also encompasses genetically engineered and/or otherwise modified forms of immunoglobulins, such as intrabodies, peptibodies, chimeric antibodies, fully human antibodies, humanized antibodies, and heteroconjugate antibodies, multispecific, e.g., bispecific, antibodies, diabodies, triabodies, and tetrabodies, tandem di-scFv, tandem tri-scFv. In a specific embodiment, the antibody fragment is a scFv. Unless otherwise stated, the term “Ab fragment” should be understood to encompass functional antibody fragments thereof. A portion of an Ab fragment that comprises a structure that enables specific binding to an antigen is referred to as “antigen-binding Ab fragment,” “AB domain,” “antigen-binding region,” or “antigen-binding region” of the Ab fragment.

An “isolated” biological component (such as an isolated protein, nucleic acid, vector, or cell) refers to a component that has been substantially separated or purified away from its environment or other biological components in the cell of the organism in which the component naturally occurs, for instance, other chromosomal and extra-chromosomal DNA and RNA, proteins, and organelles. Nucleic acids and proteins that have been “isolated” include nucleic acids and proteins purified by standard purification methods. The term also embraces nucleic acids and proteins prepared by recombinant technology as well as chemical synthesis. An isolated nucleic acid or protein can exist in substantially purified form, or can exist in a non-native environment such as, for example, a host cell.

The term “mammal” refers to any mammal, including, but not limited to, mammals of the order Rodentia, such as mice and hamsters, and mammals of the order Logomorpha, such as rabbits. The mammals may be from the order Carnivora, including Felines (cats) and Canines (dogs). The mammals may be from the order Artiodactyla, including Bovines (cows) and Swines (pigs) or of the order Perssodactyla, including Equines (horses). The mammals may be of the order Primates, Ceboids, or Simoids (monkeys) or of the order Anthropoids (humans and apes).

“Neurodegenerative diseases” are a heterogeneous group of disorders that are characterized by the progressive degeneration of the structure and function of the central nervous system or peripheral nervous system. Exemplary neurodegenerative diseases include but are not limited to Parkinson's disease (PD), Alzheimer's disease (AD), multiple sclerosis (MS), Lewy body disease or Lewy body dementia (LBD), progressive supranuclear palsy (PSP), multiple system atrophy (MSA), amyotrophic lateral sclerosis (ALS) or motor neurone diseases (MND), Huntington's Disease (HD), spinocerebellar ataxia (SCA), Friedreich's ataxia (FA), spinal muscular atrophy (SMA), and prion disease such as Creutzfeldt-Jakob disease (CJD).

The term “nucleic acid” and “polynucleotide” refer to RNA or DNA that is linear or branched, single or double stranded, or a hybrid thereof. The term also encompasses RNA/DNA hybrids. The following are non-limiting examples of polynucleotides: a gene or gene fragment, exons, introns, mRNA, tRNA, rRNA, ribozymes, cDNA, recombinant polynucleotides, branched polynucleotides, plasmids, vectors, isolated DNA of any sequence, isolated RNA of any sequence, nucleic acid probes and primers. A polynucleotide may comprise modified nucleotides, such as methylated nucleotides and nucleotide analogs, uracil, other sugars and linking groups such as fluororibose and thiolate, and nucleotide branches. The sequence of nucleotides may be further modified after polymerization, such as by conjugation, with a labeling component. Other types of modifications included in this definition are caps, substitution of one or more of the naturally occurring nucleotides with an analog, and introduction of means for attaching the polynucleotide to proteins, metal ions, labeling components, other polynucleotides or solid support. The polynucleotides can be obtained by chemical synthesis or derived from a microorganism. The term “gene” is used broadly to refer to any segment of polynucleotide associated with a biological function. Thus, genes include introns and exons as in genomic sequence, or just the coding sequences as in cDNAs and/or the regulatory sequences required for their expression. For example, gene also refers to a nucleic acid fragment that expresses mRNA or functional RNA, or encodes a specific protein, and which includes regulatory sequences.

The term “pharmaceutically acceptable excipient,” “pharmaceutical excipient,” “excipient,” “pharmaceutically acceptable carrier,” “pharmaceutical carrier,” or “carrier” as used herein refers to compounds or materials conventionally used in pharmaceutical compositions during formulation and/or to permit storage. Excipients included in the formulations will have different purposes. Examples of generally used excipients include, without limitation: saline, buffered saline, dextrose, water-for-infection, glycerol, ethanol, and combinations thereof, stabilizing agents, solubilizing agents and surfactants, buffers and preservatives, tonicity agents, bulking agents, and lubricating agents.

The term “recombinant” means a polynucleotide, a protein, a cell, and so forth with semi-synthetic or synthetic origin which either does not occur in nature or is linked to another polynucleotide, a protein, a cell, and so forth in an arrangement not found in nature.

The term “subject” as used herein may be any living organisms, preferably a mammal. In some embodiments, the subject is a primate such as a human. In some embodiments, the primate is a monkey or an ape. The subject can be male or female and can be any suitable age, including infant, juvenile, adolescent, adult, and geriatric subjects. In some examples, the patient or subject is a validated animal model for disease and/or for assessing toxic outcomes. The subject may also be referred to as “patient” in the art. The subject may have a disease or may be healthy.

The term “subject at increased risk of developing PD (or other neurologic disease associated with neural inflammation)” as used herein may be any living organism, preferably a mammal, and most preferably a primate such as a human that is at increased risk of developing PD (or other neurologic disease associated with neural inflammation) because of a family history, environmental factors, one or more concussions among other risk factors.

The term “subject having PD (or other neurologic disease associated with neural inflammation)” or “a subject determined to be at increased risk of developing PD (or other neurologic disease associated with neural inflammation)” as used herein may be any living organism, preferably a mammal, and most preferably a primate such as a human that has or is at increased risk of developing PD (or other neurologic disease associated with neural inflammation) who is determined to be at increased risk of developing PD (or other neurologic disease associated with neural inflammation) because of the increased or decreased expression of a gene the expression of which correlates with PD (or other neurologic disease associated with neural inflammation), e.g., increased expression of BRI3 on monocytes compared to BRI3 expression by normal controls.

The term “scFv,” “single-chain Fv,” or “single-chain variable fragment” refers to a fusion protein comprising at least one antibody fragment comprising a variable region of a light chain and at least one antibody fragment comprising a variable region of a heavy chain, wherein the light and heavy chain variable regions are contiguously linked, e.g., via a synthetic linker, e.g., a short flexible polypeptide linker, and capable of being expressed as a single chain polypeptide, and wherein the scFv retains the specificity of the intact antibody from which it is derived. Unless specified, as used herein an scFv may have the VL and VH variable regions in either order, e.g., with respect to the N-terminal and C-terminal ends of the polypeptide, the scFv may comprise VL-linker-VH or may comprise VH-linker-VL. The linker may comprise portions of the framework sequences. In scFvs, the heavy chain variable domain (HC V, HCV, or VH) may be placed upstream of the light chain variable domain (LC V, LCV, or VL), and the two domains may optionally be linked via a linker (for example, the G4S X3 linker). Alternatively, the heavy chain variable domain may be placed downstream of the light chain variable domain, and the two domains may optionally be linked via a linker (for example, the G4S X3 linker).

As used herein, the term “treat,” “treatment,” or “treating” generally refers to the clinical procedure for reducing or ameliorating the progression, severity, and/or duration of a disease or of a condition, or for ameliorating one or more conditions or symptoms (preferably, one or more discernible ones) of a disease. The disease to be treated may be, for example, PD, but may also treat other neurodegenerative diseases that cause a similar condition and/or symptom to that of PD. Therefore, the treatment method according to the present disclosure may also treat, a fibrotic disease, for example, pulmonary fibrosis, an interstitial lung disease, cystic fibrosis, chronic obstructive pulmonary disease, sarcoidosis, an allergic airway disease, hepatic fibrosis, or cardiac fibrosis. The condition to be treated by a method according to the present invention may be, for example, fibrosis, oxidative stress, or inflammation. In specific embodiments, the effect of the “treatment” may be evaluated by the amelioration of at least one measurable physical parameter of a disease, resulting from the administration of one or more therapies (e.g., anti-BRI3 agent, and in some cases in combination with another therapy). The parameter may be, for example, gene expression profiles, the mass of disease-affected tissues, inflammation-associated markers, fibrosis-associated markers, the number or frequency of disease-associated cells, the presence or absence of certain cytokines or chemokines or other disease-associated molecules, and may not necessarily discernible by the patient. In other embodiments “treat”, “treatment,” or “treating” may result in the inhibition of the progression of a disease, either physically by, e.g., stabilization of a discernible symptom, physiologically by, e.g., stabilization of a physical parameter, or both. In other embodiments the terms “treat”, “treatment” and “treating” refer to the reduction or stabilization of inflammatory or fibrotic tissue. Additionally, the terms “treat,” and “prevent” as well as words stemming therefrom, as used herein, do not necessarily imply 100% or complete cure or prevention. Rather, there are varying degrees of treatment effects or prevention effects of which one of ordinary skill in the art recognizes as having a potential benefit or therapeutic effect. In this respect, the inventive methods can provide any amount of any level of treatment or prevention effects of a disease in a mammal. Furthermore, the treatment or prevention provided by the inventive method can include treatment or prevention of one or more conditions or symptoms of the disease being treated or prevented. Also, for purposes herein, “prevention” can encompass delaying the onset of the disease, or a symptom or condition thereof.

As disclosed previously, the present invention provides methods of treating and/or preventing and/or delaying the onset of neurodegenerative diseases such as Parkinson's disease (PD), methods of treating and/or preventing neural inflammation associated with neurodegenerative diseases such as PD, methods of diagnosing neurodegenerative diseases such as PD, methods of determining the severity and/or stage of neurodegenerative diseases such as PD, methods of determining the inflammatory status or levels in neurodegenerative diseases such as PD, methods of determining whether a therapy for neurodegenerative diseases such as PD is effective in a subject, methods of screening for a therapeutic agent for neurodegenerative diseases such as PD, and methods of predicting whether a subject with a neurodegenerative disease such as PD will respond to a therapy. The present invention further relates to agents that suppress, inhibit, block, and/or antagonize BRI3, compositions comprising such an agent, and kits for detecting expression of BRI3.

In some embodiments such treatment or prophylaxis may include the administration or use of one or more other agents or drugs or other treatments useful for treatment or prophylaxis of the neurodegenerative disease or neuroinflammation, e.g., one or more other agents or treatments useful for treatment or prophylaxis of PD.

These other agents or treatments may be administered separately or in combination with the inventive methods of treatment or prevention. In particular such agents or drugs or other treatments may include one or more of the following:
(i) levodopa alone or in combination with carbidopa and benserazide (which are dopa decarboxylase inhibitors that do not cross the blood-brain barrier and inhibit the conversion of levodopa to dopamine outside the brain, reducing side effects and improving the availability of levodopa for passage into the brain). These drugs optionally may comprise by way of example controlled-release (CR) versions of levodopa or extended-release levodopa formulations, oral, longer-acting formulations, as well as inhaled or transdermal formulations.
(ii) COMT or COMT inhibitors Catechol-O-methyltransferase (COMT) optionally may be used in conjunction with levodopa/carbidopa when a person is experiencing the “wearing off phenomenon” with their motor symptoms. COMT inhibitors which may be used to treat PD and end-of-dose motor fluctuations include by way of example opicapone, entacapone, and tolcapone.
(iii) Other Dopamine agonists such as pergolide, pramipexole, ropinirole, piribedil, cabergoline, apomorphine, bromocriptine, and lisuride.
(iii) MAO-Inhibitors such as safinamide, selegiline and rasagiline which increase the amount of dopamine in the basal ganglia by inhibiting the activity of monoamine oxidase B, an enzyme that breaks down dopamine.
(v) Other drugs such as amantadine and anticholinergics such as quetiapine or pimavanserin for psychosis, cholinesterase inhibitors for dementia, and modafinil, doxepin and rasagline for excessive daytime sleepiness.
(v) Combination drugs such as Nourianz (istradefylline), a recently approved add-on treatment to levodopa/carbidopa in adult patients with Parkinson's disease (PD) experiencing “off” episodes.
(vi) Other Treatments (non-drug treatments) such as other gene therapies, cell-based therapies, and surgery. Surgery for PD includes lesional and deep brain stimulation (DBS). Target areas for DBS or lesions include the thalamus, globus pallidus, or subthalamic nucleus. DBS involves the implantation of a medical device called a neurostimulator, which sends electrical impulses to specific parts of the brain. Other, less common surgical therapies involve intentional formation of lesions to suppress overactivity of specific subcortical areas. For example, pallidotomy involves surgical destruction of the globus pallidus to control dyskinesia.

Four areas of the brain have been treated with neural stimulators in PD, i.e., the globus pallidus interna, thalamus, subthalamic nucleus, and pedunculopontine nucleus. DBS of the globus pallidus interna improves motor function, while DBS of the thalamic DBS improves tremor.

Examples are provided below to illustrate the present invention. These examples are not meant to constrain the present invention to any particular application or theory of operation.

### EXAMPLES

### Example 1: Identification of Genes Wherein Expression is Modified in PD Patients

Peripheral immune cells are executors of the immune inflammatory response. Based thereon we hypothesized that if innate immune inflammatory responses correspond with the brain pathology in PD or neural inflammation, peripheral immune cells and/or peripheral immune responses potentially represent an easily sampled surrogate for detection of neural inflammation occurring in association with PD and potentially other neural diseases associated with neural inflammation.

A growing number of studies implicate monocyte dysregulation in PD, but there is still not enough known about the PD-associated monocyte state. We leveraged newly available single-cell transcriptomic technologies to better characterize monocytes in PD in order to identify gene expression signatures and pathways of interest for the management of the disease. Using this approach, we integrated and analyzed over 17,000 peripheral blood mononuclear cells (PBMCs) obtained from a small cohort of PD patients and controls. Deep sequencing at single cell resolution characterizing monocytes revealed panels of genes differentially expressed in subsets of PD monocytes compared with controls. We validated BRI3, a gene consistently upregulated in the CD16 subset of PD monocytes, using biochemical techniques. As shown herein we identified elevated BRI3 protein expression in activated cultured human monocytes and in the extracellular vesicle fraction of PD patient plasma. We inactivated BRI3 in the THP1 monocyte cell line and demonstrate that BRI3 loss reduces inflammatory cytokine secretion in cultured monocytes. Taken together the study supports the existence of a PD-specific monocyte cell state and identifies BRI3 as a novel indicator, and likely regulator, of innate immune perturbation in PD.

Methods

Rapidly emerging technology now allows for RNA-sequencing at single-cell resolution (scRNA-Seq) (Hwang, B., J. H. Lee, and D. Bang, Single-cell RNA sequencing technologies and bioinformatics pipelines. Exp Mol Med, 2018. 50(8): p. 96). Data analysis protocols are continuously evolving with first-generation studies focused on single or paired sample comparisons identifying new cellular subtypes (Ximerakis, M., et al., Single-cell transcriptomic profiling of the aging mouse brain. Nat Neurosci, 2019. 22(10): p. 1696-1708). and transcriptomic states (Reitman, Z. J., et al., Mitogenic and progenitor gene programmes in single pilocytic astrocytoma cells. Nat Commun, 2019. 10(1): p. 3731). We also identified a small cohort of patients suitable for analysis as an integrated dataset in order to make determinations regarding the state of peripheral immune cells in PD. This cohort was limited to male patients for three reasons: (1) in order to remove variation due to sex chromosome dosage; (2) to circumvent the now recognized confound of heterogeneous X inactivation across individual female cells (Garieri, M., et al., Extensive cellular heterogeneity of X inactivation revealed by single-cell allele-specific expression in human fibroblasts. Proc Natl Acad Sci USA, 2018. 115(51): p. 13015-13020); and (3) We were able to identify 2 healthy controls and 3 PD male volunteers of similar age whose medical history did not indicate co-morbidities likely to influence the innate immune system in a reasonable timeframe (Table 1). PD volunteers represented a spectrum of PD progression with time since diagnosis ranging from 1 to 6 years and UPDRS scores from 8 to 56. Patients varied in their clinical medication regimes that included 1 patient using oral levodopa/carbidopa, 1 prescribed ropinirole and rasagiline, and one early-stage participant taking part in a clinical study involving a cAbI inhibitor. Patients all self-report use of over the counter anti-inflammatory medications of various brands.

TABLE 1

| | Controls (2 males) | PD (3 males) | |
|---|---|---|---|
| Ages (years) | 60.5 (0.7) | 71.4 | (13.2) |
| Ethnicity (% non-hispanic) | 100 | 100 | |
| Disease duration (years) | N/A | 3.5 | (2.0) |
| UPDRS (total) | N/A | 29.7 | (24.3) |
| Dopamine replacement (%) | N/A | 33.3 | (57.7) |
| Dopamine agonist (%) | N/A | 33.3 | (57.7) |
| Dopamine anti-degradation (%) | N/A | 33.3 | (57.7) |

Values are depicted as mean (SD) unless otherwise noted.
Dopamine replacement: carbidopa/levodopa; dopamine agonist; ropinirole, pramipexole; dopamine anti-degradation; rasagiline, entacapone

Clinical heterogeneity is observed in PD patients as the result of aging, lifestyle, and environmental factors, among others. A key advance made by authors is the creation of an original computational pipeline, drawing together multiple programming strategies to integrate the data and allow for the interrogation of changes in gene expression in individual cell types. Preprocessing and quantification of gene expression levels was performed using CellRanger v3.1. Briefly, reads associated with each sample were aligned by indexing to GRCh38 (GENCODE v.24) using STAR v.2.5.0, trimming read 2 to remove 3′ poly(A) tails (>7 A's) and discarding fragments with fewer than 24 remaining nucleotides as described (Yuan, J., et al., “Single-cell transcriptome analysis of lineage diversity in high-grade glioma”, Genome Med, 2018. 10(1): p. 57). Aligned reads were then assigned to cell-specific barcodes and PCR duplicates removed by counting Unique Molecular Identifier (UMI) sequences. 1 base-pair error correction will be applied to both UMIs and cell-barcodes to account for sequencing errors. Per-gene UMI counts will be used to generate a preliminary gene expression matrix. Subsequently, outputs from CellRanger were loaded into the R statistical programming environment (v 3.6.1) using the Seurat v3 R package (Stuart, T., et al., “Comprehensive Integration of Single-Cell Data”, Cell, 2019. 177(7): p. 1888-1902 e21) and subjected to additional quality control procedures. Cells exhibiting high proportions of reads mapping to the mitochondrial genome or low expression complexity (based on total UMI count and number of detected features) were removed from downstream analysis (Szabo, P. A., et al., “Single-cell transcriptomics of human T cells reveals tissue and activation signatures in health and disease”, Nat Commun, 2019. 10(1): p. 4706). Expression of key cell-cycle regulators determined potential variation across the dataset attributable to cell-cycle phase. If determined to explain a significant proportion of gene expression differences, variability attributable to cell cycle stage was statistically regressed out during the normalization step. Normalization of raw counts was performed using the recently described SCTransform method implemented in Seurat v3. Finally, corresponding gene expression profiles shared across cells from independent samples will be identified using recently developed canonical correlation analysis (CCA)-based method implemented in Seurat v3, and used to perform dataset integration and batch correction, ultimately producing a single dataset to be used for all downstream analyses (Hafemeister, C. and R. Satija, Normalization and variance stabilization of single-cell RNA-seq data using regularized negative binomial regression. Genome Biol, 2019. 20(1): p. 296).

Results

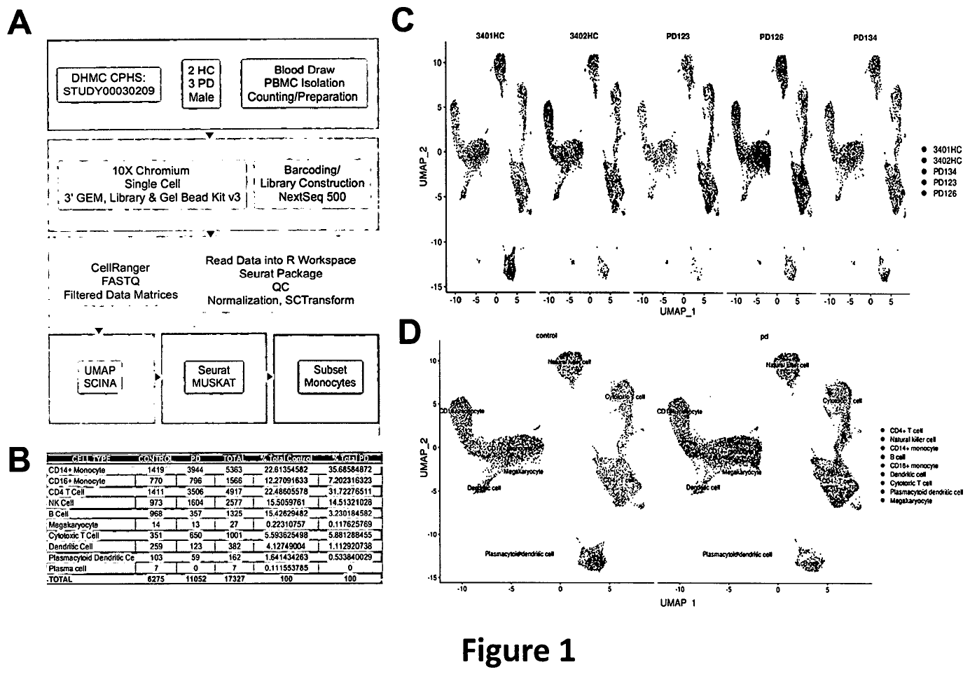

High variability is a concern in the analysis of transcriptomic state in highly reactive human cell populations in aging individuals diagnosed with complex and slowly progressive diseases like PD and treated using different therapeutic regimes. We collected whole blood from five volunteers (n=2 healthy control, 3 PD) using clinical methodologies consistent with the widely-accepted protocols laid out by the Parkinson's Progression Marker's Initiative (“The Parkinson Progression Marker Initiative (PPMI)”, Prog Neurobiol, 2011. 95(4): p. 629-35) making minor changes in the blood draw procedure for optimal PMBC isolation. The overall workflow is presented in FIG. 1A. Single cell suspension containing purified PBMCs were processed with Chromium Single Cell B Chip (10× Genomics) and pooled libraries were sequenced using the NextSeq500 instrument. Preprocessed data from experimental cells selected as described above was subject to dimensionality reduction using uniform manifold approximation and projection (UMAP) (Becht, E., et al., “Dimensionality reduction for visualizing single-cell data using UMAP”, Nat Biotechnol, 2018) to aggregate cells of similar transcriptional profiles. Resulting cell clusters were assigned cellular identities using the prior knowledge-based SCINA algorithm (Zhang, Z., et al., “SCINA: A Semi-Supervised Subtyping Algorithm of Single Cells and Bulk Samples”, Genes (Basel), 2019. 10(7)). As expected, we collected more cells from the larger PD group, but this discrepancy did not influence cell clustering during data integration (FIGS. 1B and C). We achieved reasonable segregation of the major subcategories anticipated in PBMC populations (FIG. 1D). No cluster was driven by over-representation of cells from a single individual (FIGS. 1C and D). Close inspection of the data however indicated changes in the proportions of cell populations in PD compared with health controls. Despite analyzing more cells overall, in PD patients we identified relatively fewer CD16 monocytes, B cells, and dendritic cells compared with controls. In contrast, we detected increased numbers of CD14 monocytes and CD4 T cells in PD patients compared with controls (FIGS. 1B and D). The finding of changes in the CD14/CD16 ratio in PD is consistent with previous reports analyzing immune cells in larger cohorts indicating increased CD14 monocytes in PD and decreases in the CD16 subset (Grozdanov, V., et al., “Inflammatory dysregulation of blood monocytes in Parkinson's disease patients”, Acta Neuropathol, 2014. 128(5): p. 651-63). Data indicate that the anticipated cell populations were identified and sufficiently represented for each patient the integrated dataset despite the clinical and sampling variability.

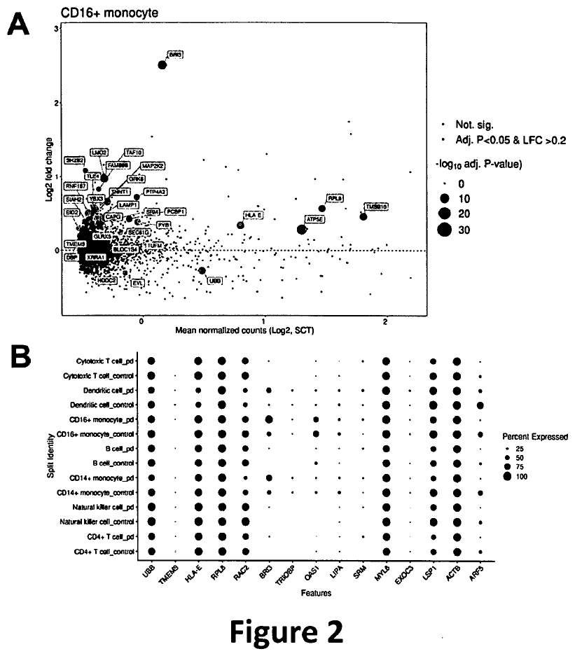

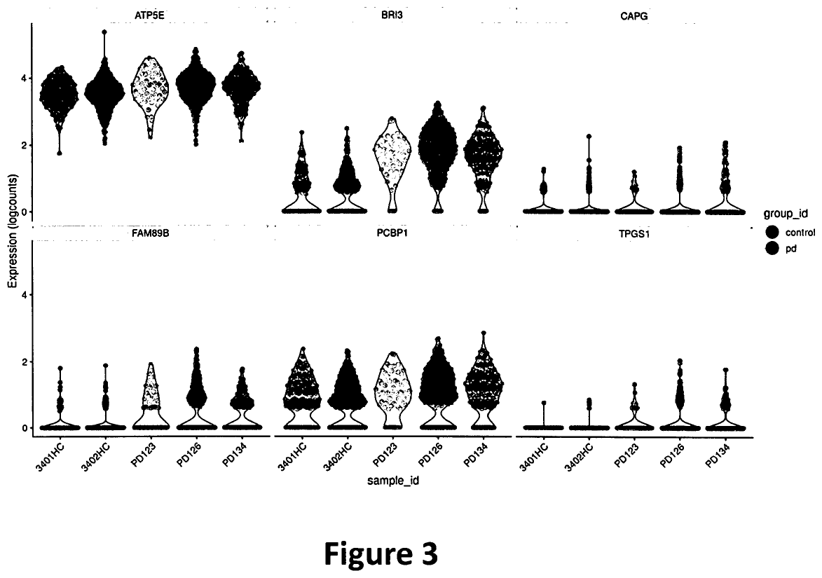

Methodologies for analysis of differential gene expression in scRNA-Seq datasets continue to evolve. Challenges arising based on the sparsity of the data are augmented by the clinical complexity of diseases like PD, variation in the number of samples available per group, number of cells and cell subpopulations identified in individuals, and the magnitude of differential expression. To overcome these challenges we used a recently described analysis pipeline that accounts for cell and sample-level changes, utilizing mixed-modeling strategies in data normalized by an internal anchored reference dataset (muscat R package, (Helena L. Crowell, C. S., Pierre-Luc Germain, Daniela Calini, Ludovic Collin, Catarina Raposo, Dheeraj Malhotra, Mark D. Robinson, “On the discovery of subpopulation-specific state transitions from multi-sample multi-condition single-cell RNA sequencing data”, bioRxiv, 2020)). Among genes observed to be significantly altered in PD, we identified several genes of interest that withstood this rigorous statistical analysis in the CD16 subset (FIG. 2A-B). The mixed modeling analysis strategy we employed was designed expressly to normalize multi-sample, multi-group, multi-(cell-)subpopulation scRNA-seq data (Helena L. Crowell, C. S., Pierre-Luc Germain, Daniela Calini, Ludovic Collin, Catarina Raposo, Dheeraj Malhotra, Mark D. Robinson, “On the discovery of subpopulation-specific state transitions from multi-sample multi-condition single-cell RNA sequencing data”, bioRxiv, 2020); however, previous reports highlight substantial variability in monocyte gene expression in PD (Schlachetzki, J. C. M., et al., “A monocyte gene expression signature in the early clinical course of Parkinson's disease”, Sci Rep, 2018. 8(1): p. 10757). To further confirm differentially expressed genes in our cohort, we evaluated genes of the highest significance individually across all 5 individuals. Analysis of gene expression in the five individuals highlighted variability in the total number of cells detected with high levels of expression of the genes identified as differentially expressed using the mixed modeling strategy. We evaluated CD16 monocytes and found changes that were more robust than we had observed analyzing CD14 monocytes. Increased expression of ATP5E, BRI3, PCBP, and FAM89B (encoding ATP synthase F1 subunit epsilon, brain protein I3, poly(rC) binding protein 1, and family with sequence similarity 89 member B, respectively) were consistently observable in PD patients compared with controls (FIG. 3). These findings indicate several genes whose expression is convincingly altered in PD compared with controls. Data indicate robust alterations in gene expression in the CD16 non-classical or “patrolling” monocyte subpopulation, typically associated with the modulation of inflammatory processes, maintenance of vascular endothelial homeostasis, responding to tissue injury and chronic inflammatory disease (Narasimhan, P. B., et al., “Nonclassical Monocytes in Health and Disease”, Annu Rev Immunol, 2019. 37: p. 439-456). The findings support previous reports indicating detectable alterations in the state of PD monocytes (Grozdanov, V., et al., “Inflammatory dysregulation of blood monocytes in Parkinson's disease patients”, Acta Neuropathol, 2014. 128(5): p. 651-63; Schlachetzki, J. C. M., et al., “A monocyte gene expression signature in the early clinical course of Parkinson's disease”, Sci Rep, 2018. 8(1): p. 10757; and Nissen, S. K., et al., “Alterations in Blood Monocyte Functions in Parkinson's Disease”, Mov Disord, 2019. 34(11): p. 1711-1721), and extend these studies using high-resolution transcriptomics to identify new genes of interest for delineation of the complexities of monocyte dysregulation in PD.

As shown herein we have discovered several genes elevated in PD across multiple cell types. Among them, we focused on the robust and reproducible enrichment of BRI3 transcript in a large number of CD16 monocytes PD patients (FIG. 3). BRI3 reportedly is expressed on some hematopoietic cells and is in a list of genes identified using screening strategies to define changes related to hematopoietic maturation (Mello, F. V., et al., “Maturation-associated gene expression profiles along normal human bone marrow monopoiesis”, Br J Haematol, 2017. 176(3): p. 464-474). Mello also cites two earlier publications tangentially relating BRI3 biology to the cytokine response. In the first article published in 2003, the authors reportedly observed that the suppression of BRI3 expression resulted in resistance to tumor necrosis factor-induced cell death in cultured fibroblasts (Wu et al., “bri3, a novel gene, participates in tumor necrosis factor-a-induced cell death”, Biochem Res. Comm. 311(2) pp. 528-34). In a second publication, published in 2018, the authors allege that BRI3 may interact with IFITM3, a modulator of the interferon response, in a yeast to-hybrid assay (Akiva et al., “Identification of IFITM3 and MGAT1 as novel interaction partners of BRI3 by yeast two-hybrid screening”, Turk J Biol., 42(6): 463-470).

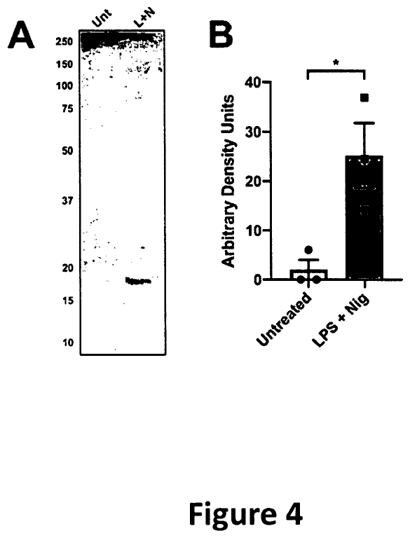

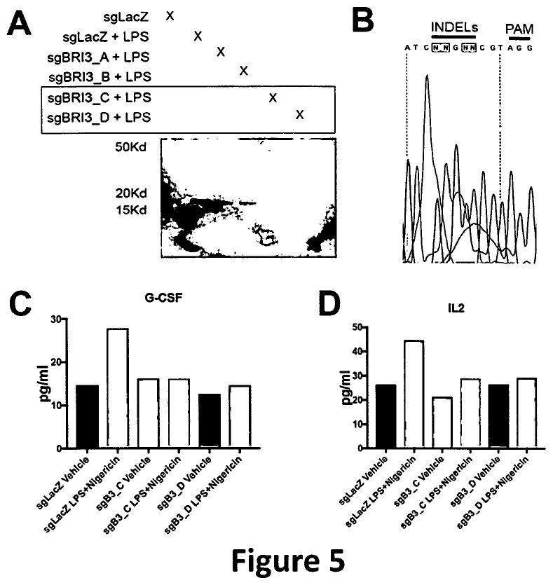

By contrast, our results suggest that BRI3 has an immune modulating function in PD innate immune cells, especially in monocytes and closely-related CNS microglia. Our supposition that BRI3 functions in innate immune cells like monocytes is supported by our biological validation of scRNA-Seq studies in which we have observed that BRI3 was inducible in cultured human monocytes exposed to inflammatory insult (cultured in the presence of LPS and nigericin) (FIG. 4A-B) and that BRI3 inactivation using CRISPR/CAS9 reduced the cytokine response in the same monocyte cell line (FIG. 5A-D).

### Example 2: Diagnosis of PD

Currently there is no definitive test for diagnosing PD. Rather, diagnosis is generally made based on symptoms, such as tremor, impaired movements and balance, changes in speech and writing, tests that rule out other neurological diseases, and response to carbidopa-levodopa.

According to the present discovery, PD or an increased risk for developing PD may be diagnosed based on the aberrant (increased) expression of BRI3 and, in certain examples, other genes related to the monocyte cell state. Subjects suspected to have PD or those having an increased risk for developing PD, e.g., because of a family history or environmental factors (e.g., prolonged exposure to pesticides and herbicides; Vietnam-era exposure to Agent Orange; prolonged exposure to heavy metals, detergents and solvents) will be tested. For example, subjects who show at least one symptom that may be of PD and/or subjects at risk for developing PD (e.g., genetically predisposed subject) will be tested. BRI3 elevation or related changes in the monocyte state may indicate PD, risk of developing PD, occupation, lifestyle, or environmental exposure that increase risk of developing PD, or a clinical condition or lifestyle with the potential to accelerate the progression of PD.

Peripheral blood samples will be collected from the subjects and peripheral blood mononuclear cells (PBMCs) will be isolated using a standard protocol. Additionally, or alternatively, cerebrospinal fluid (CSF) will be collected from the subject and cell contents will be isolated using a standard protocol.

### Transcript Analyses

In one test, transcript analyses on the PBMCs and/or CSF cells will be performed. For example, single cells will be first obtained and single-cell RNA sequencing, single-cell quantitative PCR (qPCR), or single-cell nanostring may be used. Alternatively, cells may be presorted into different cell types (e.g., immune cell types) and then gene expression may be determined via quantitative PCR (qPCR).

Transcripts of genes such as BRI3, FAM89B, PCBP1, ATP5F1E (or ATP5E), SH2B2, LMO2, TAF10, MAP2K2, TLE4, GRK6, RNF187, TNNT1, PTP4A2, SIAH2, YBX3, LAMP1, RPL8, EID2, CAPG, SRM, TMSB10, FYB, HLA-E, GLRX5, SEC61G, TMEM9, DBP, XRRA1, BLOC1S4, TUFM, HDDC2, EVL, UBB, RAC2, OAS1, LSP1, or ARF5 in various cell types may be analyzed. Alternatively, at least the transcript levels of BRI3 may be analyzed. If BRI3 expression is significantly higher than that of a healthy control or than a standard level, for example in monocytes (e.g., CD16⁺ monocytes and/or CD14⁺ monocytes), the subject will be diagnosed as having PD.

Transcript levels of other genes measured may also be taken into account towards diagnosis. For example, upregulation of one or more of FAM89B, PCBP1, ATP5F1E (or ATP5E), SH2B2, LMO2, TAF10, MAP2K2, TLE4, GRK6, RNF187, TNNT1, PTP4A2, SIAH2, YBX3, LAMP1, RPL8, EID2, CAPG, SRM, TMSB10, FYB, HLA-E, GLRX5, SEC61G, TMEM9, DBP, XRRA1, BLOC1S4, or TUFM and/or downregulation of one or more of HDDC2, EVL, UBB, RAC2, OAS1, LSP1, or ARF5 may be further included as a criteria for diagnosing PD. For instance, downregulation of LSP1 in cytotoxic T cells (or CD8⁺ T cells), CD16⁺ monocytes, CD14⁺ monocytes, and/or NK cells, and/or downregulation in ARF5 in cytotoxic T cells (or CD8+ T cells), dendritic cells, CD16⁺ monocytes, B cells, CD14⁺ monocytes, NK cells, and/or CD4⁺ T cells may be further included as a criteria for diagnosing PD. Changes in the population of immune cells may also be taken into account towards diagnosis. For example, reduction in CD16⁺ monocytes, B cells, and/or dendritic cells and/or increase in CD14⁺ monocytes and/or CD4⁺ T cells may be further included as a criteria for diagnosing PD.

### Protein Expression Analyses

In another test, PBMCs and/or CSF cells be stained, e.g., with antibodies, for various immune cell markers (e.g., one or more of CD45, CD45RO, CD45RA, CD1a, CD3, CD4, CD8, CD11b, CD11c, CD14, CD16, CD19, CD20, CD34, CD40, CD56, CD64, CD68, CD71, CD80, CD83, CD86, CD94, CCR5, FceRIa, HLA-DR) and for BRI3, and the protein expression levels will be determined by, e.g., flowcytometry. If BRI3 expression is significantly higher than that of a healthy control or than a standard level, for example in monocytes (e.g., CD16⁺ monocytes and/or CD14⁺ monocytes) and/or dendritic cells, the subject will be diagnosed as having PD.

Changes in the expression of other genes may also be taken into account towards diagnosis. For example, downregulation of LSP1 in cytotoxic T cells (or CD8⁺ T cells), CD16⁺ monocytes, CD14⁺ monocytes, and/or NK cells, and/or downregulation in ARF5 in cytotoxic T cells (or CD8⁺ T cells), dendritic cells, CD16⁺ monocytes, B cells, CD14⁺ monocytes, NK cells, and/or CD4⁺ T cells may be further included as a criteria for diagnosing PD. Changes in the population of immune cells may also be taken into account towards diagnosis. For example, reduction in CD16⁺ monocytes, B cells, and/or dendritic cells and/or increase in CD14⁺ monocytes and/or CD4⁺ T cells may be further included as a criteria for diagnosing PD.

### Extracellular-Released Vesicle Analysis

Extracellular-released vesicles (EVs), such as exosomes and microvesicles (MVs), are released from cell membranes and contain various biological materials such as nucleic acids (e.g., RNA) and proteins of the cell of origin. Recently, neuronal exosomes are shown to be a great tool for detecting important disease information in a subject (Jiang C. et al., J. Neurol Neurosurg Psychiatry, 2020 Jul; 91(7):720-729. doi: 10.1136/jnnp-2019-322588. Epub 2020 Apr 9). According to the present discovery, microglia express high levels of BRI3 in PD patients, and therefore PD or an increased risk of developing PD will be diagnosed based on an increased level of BRI3 in the EVs (exosomes and/or MVs).

Blood samples and/or CSF samples will be obtained from subjects. Exosomes will be isolated using a standard protocol (e.g. ultracentrifugation and size exclusion chromatography) and lysed. Exosomal RNA and proteins will be extracted using a standard protocol. BRI3-encoding RNA and/or BRI3 protein will be quantified using a standard method (e.g., qPCR for RNA and enzyme-linked immunosorbent assay (ELISA) for protein).

### Neuroimaging

In current diagnosis of PD, imagining tests such as an MRI, ultrasound, and positron emission tomography (PET) scans are used to rule out other neurodegenerative diseases. According to the present discovery, microglia in the brain may express high levels of BRI3. Therefore, incorporating BRI3 imaging during such imaging tests will allow for more accurate diagnosis of PD. Various ligand-specific brain image technologies are available (e.g., Sehlin D. et al., “Engineered antibodies: new possibilities for brain PET?”, Eur J. Nucl Med Mol Imaging, 2019 December; 46(13):2848-2858).

In some examples, a subject will receive an antibody fragment-based BRI3-specific agent (e.g., radiolabeled anti-BRI3 F(ab′)2 fragment conjugated to an anti-transferrin receptor (TfR) antibody) and then be subject to PET scan. If the patient brain is stained positive with the agent, the subject will be diagnosed with PD.

When diagnosing, regardless of whether the diagnosis is based on the transcriptome, protein expression, or exosomal analyses, or neuroimaging, the stage and/or disease progression may be further determined based on the degree of gene upregulation (e.g. of BRI3) and/or downregulation and/or the degree of changes in the immune population. Furthermore, these analyses are applicable not only to diagnosis of PD but also to determination of whether a subject has inflammation associated with a neurodegenerative disease such as PD.

Once diagnosed with PD and/or neural inflammation, the subject may be administered with one or more therapeutics for PD and/or neural inflammation. Non-limiting examples of the therapeutic include levodopa, carbidopa, a dopamine agonist (e.g., pramipexole, ropinirole, rotigotine, apomorphine), a monoamine oxidase B (MAO B) inhibitor (e.g., selegiline, rasagiline, safinamide), a catechol O-methyltrasnferase (COMT) inhibitor (e.g., entacapone, tolcapone), an anticholinergic (e.g., benztropine), amantadine, an analgesic, a corticosteroid, an anti-inflammatory, a microglial suppressor, a neuroregenerating agent (e.g., vitamin B12, chorionic gonadotropin), a neurohormone (e.g., oxytocin), or any combination thereof, further optionally comprising administering deep brain stimulation (DBS).

### Example 3: Detection of Disease Progression in Patients

Changes in the gene expression and/or immune cell populations are also useful in monitoring disease progression in a patient who already has a neurodegenerative disease (e.g., whether the severity and/or stage of PD has advanced, and/or inflammatory levels have increased).

Blood and/or CSF samples will be obtained from a subject contracted with a neurodegenerative disease such as PD at multiple timepoints (e.g., about once per three months, about once per 6 months, about once per year, etc). Transcript and/or protein expression of genes (e.g., BRI3) and/or immune cell population will be analyzed as described in Example 2.

If BRI3 expression as transcript and/or protein has significantly increased relative to a previous timepoint, for example in monocytes (e.g., CD16⁺ monocytes and/or CD14⁺ monocytes), it will be determined that the disease severity and/or stage has advanced and/or inflammatory levels have increased. In some cases, expression of other genes and immune cell population (proportion) will be also determined as in Example 2 and used towards determining the progression. Nucleic acid and/or protein contents in exosomes isolated from the blood and/or CSF samples may also be also analyzed and utilized toward determining disease progression.

What is claimed is:

1. A method of treating or preventing or inhibiting the onset of a neurodegenerative disease associated with neuroinflammation, comprising administering to a subject in need thereof an active agent that modifies the expression and/or function of BRI3, FAM89B, PCBP1, ATP5F1E (or ATP5E), SH2B2, LMO2, TAF10, MAP2K2, TLE4, GRK6, RNF187, TNNT1, PTP4A2, SIAH2, YBX3, LAMP1, RPL8, EID2, CAPG, SRM, TMSB10, FYB, HLA-E, GLRX5, SEC61G, TMEM9, DBP, XRRA1, BLOC1S4, TUFM, HDDC2, EVL, UBB, RAC2, OAS1, LSP1, or ARF5, or any combination thereof, optionally wherein at least the expression and/or function of BRI3 is modified (increased) in the subject, optionally wherein the neurodegenerative disease associated with neuroinflammation is Parkinson's disease (PD).

2. A method of treating or preventing or inhibiting the onset of neural inflammation associated with a neurodegenerative disease, comprising administering to a subject in need thereof an active agent that modifies the expression and/or function of BRI3, FAM89B, PCBP1, ATP5F1E (or ATP5E), SH2B2, LMO2, TAF10, MAP2K2, TLE4, GRK6, RNF187, TNNT1, PTP4A2, SIAH2, YBX3, LAMP1, RPL8, EID2, CAPG, SRM, TMSB10, FYB, HLA-E, GLRX5, SEC61G, TMEM9, DBP, XRRA1, BLOC1S4, TUFM, HDDC2, EVL, UBB, RAC2, OAS1, LSP1, or ARF5, or any combination thereof, optionally wherein at least the expression and/or function of BRI3 is modified.

3. The method according to claim 1 or 2, wherein the active agent reduces the expression and/or function of BRI3, FAM89B, PCBP1, ATP5F1E (or ATP5E), SH2B2, LMO2, TAF10, MAP2K2, TLE4, GRK6, RNF187, TNNT1, PTP4A2, SIAH2, YBX3, LAMP1, RPL8, EID2, CAPG, SRM, TMSB10, FYB, HLA-E, GLRX5, SEC61G, TMEM9, DBP, XRRA1, BLOC1S4, or TUFM, or any combination thereof, optionally wherein at least the expression and/or function of BRI3 is reduced, further optionally wherein the reduction is by about 25%, about 30%, about 40%, about 45%, about 50%, about 55% about 60%, about 65%, about 70%, about 75%, about 80%, about 85%, about 90%, about 95%, or about 100%.

4. The method according to any one of claims 1-3, wherein the active agent increases the expression and/or function of HDDC2, EVL, UBB, RAC2, OAS1, LSP1, or ARF5, or any combination thereof, optionally wherein the increase is by about 25%, about 30%, about 40%, about 45%, about 50%, about 55% about 60%, about 65%, about 70%, about 75%, about 80%, about 85%, about 90%, about 95%, about 100%, about 150%, about 200%, about 300%, about 400%, about 500%, about 600%, about 800%, about 900%, about 1000%, about 2000%, about 3000%, about 4000%, about 5000%, or about 10000%.

5. The method according to any one of claims 1-4, wherein the modification takes place at least in peripheral blood mononuclear cells (PBMCs), monocytes, dendritic cells, and/or central nervous system (CNS) cells, optionally wherein the monocytes are circulating monocytes, further optionally wherein the monocytes are CD16⁺ monocytes and/or CD14⁺ monocytes, and yet further optionally wherein the CNS cells comprise or are microglia.

6. The method according to any one of claims 1-5, wherein the active agent at least reduces the expression and/or function of BRI3 at least in monocytes, optionally wherein the monocytes are circulating monocytes, further optionally wherein the monocytes are CD16⁺ monocytes CD14⁺ monocytes, and/or meningeal monocytes.

7. The method according to any of claims 1-6, wherein the active agent that modifies the expression and/or function comprises a clustered Regularly Interspaced Short Palindromic Repeat (CRISPR)/Cas gene editing agent, a zinc-finger nuclease (ZFN) gene editing agent, a transcription activator-like effector nuclease (TALEN) gene editing agent, a transposase-based gene therapy, an siRNA, an shRNA, an miRNA, an aptamer, an antibody, an antigen-binding antibody fragment (e.g., scFv, Fab, Fab′, (Fab′)2), a chimeric antigen receptor (CAR)-expressing cell, a peptide, a small molecule, a polymer, an expression vector encoding a gene of interest, or any combination thereof.

8. The method according to any one of claims 1-7, wherein the active agent:
(i) comprises a CRISPR/Cas gene editing agent against BRI3;
(ii) comprises or consists of a short-guide RNA (sgRNA) selected from the oligonucleotide sequences AACTCTATCGTGGTCGTAGG, CGTCACAGGTGGGCCCGTAA, GACTACGCGTGCGGCCCGCA (depicted here in “sense” orientation),
(iii) comprises or consists of a sgRNA targeting BRI3 falling within or including any of the following genomic sequences: GAGGAAGCGACGATGCCCCAACTGTGGAGC, ACCACGATAGAGTTGGCAGGATAGCGGGTG, AGTTGGGGCATCGTCGCTTCCTCAAGGCAA, TTACGGGCCCACCTGTGACGAGGTAGGGGT, GCCCTACCCCTACCTCGTCACAGGTGGGCC, TCCTGCCAACTCTATCGTGGTCGTAGGAGG, CCCGCTATCCTGCCAACTCTATCGTGGTCG, GGGCGACTACGCGTGCGGCCCGCACGGCTA, GCCCACCTGTGACGAGGTAGGGGTAGGGCG, CCCAGGGTCTACAACATCCACAGCCGGACC, CCACGATAGAGTTGGCAGGATAGCGGGTGA, TTGGCAGGATAGCGGGTGACGGTCCGGCTG, CAGGGATACCCACCCACCATCCCAGGGTCT, CCTGGTGTTCCCTTTAAGCGAAGGTGGCTC (as annotated in Gene ID 25798, NM_015379.5, or identical BRI3 sequences in prior or future annotations),
(iv) comprises a sgRNA sequence selected from one comprising or consisting of any of the following nucleic acid sequences:
GAGGAAGCGACGATGCCCCAACTGTGGAGC
GAGGAAGCGACGATGCCCCAACTGTGGAGC
GAGGAAGCGACGATGCCCCAACTGTGGAGC
GAGGAAGCGACGATGCCCCAACTGTGGAGC
GAGGAAGCGACGATGCCCCAACTGTGGAGC
GAGGAAGCGACGATGCCCCAACTGTGGAGC
GAGGAAGCGACGATGCCCCAACTGTGGAGC
GAGGAAGCGACGATGCCCCAACTGTGGAGC
GAGGAAGCGACGATGCCCCAACTGTGGAGC
GAGGAAGCGACGATGCCCCAACTGTGGAGC
GAGGAAGCGACGATGCCCCAACTGTGGAGC
ACCACGATAGAGTTGGCAGGATAGCGGGTG
AGTTGGGGCATCGTCGCTTCCTCAAGGCAA
TTACGGGCCCACCTGTGACGAGGTAGGGGT
GCCCTACCCCTACCTCGTCACAGGTGGGCC
TCCTGCCAACTCTATCGTGGTCGTAGGAGG
CCCGCTATCCTGCCAACTCTATCGTGGTCG
GGGCGACTACGCGTGCGGCCCGCACGGCTA
GCCCACCTGTGACGAGGTAGGGGTAGGGCG
CCCAGGGTCTACAACATCCACAGCCGGACC
CCACGATAGAGTTGGCAGGATAGCGGGTGA
TTGGCAGGATAGCGGGTGACGGTCCGGCTG
CAGGGATACCCACCCACCATCCCAGGGTCT
CCTGGTGTTCCCTTTAAGCGAAGGTGGCTC
AGCGGCCGCCCGCCTACAACCTGGAGGCCG
ACAGGGATACCCACCCACCATCCCAGGGTC
TAAGCGAAGGTGGCTCCACAGTTGGGGCAT
CCAAAGGGGAAGAGGATGATGGCCAGGAAG
TGGGATGGTGGGTGGGTATCCCTGTGGGCG
CAGCAAATGAACCCAAAGGGGAAGAGGATG
TACGGGCCCACCTGTGACGAGGTAGGGGTA
CTACGCGTGCGGCCCGCACGGCTACGGCGC
GGCGGGCGGCCGCTCCTGCAGCAGCGGCTT
GACCACAAGCCGCTGCTGCAGGAGCGGCCG
CTGCCCGTCTCTGCTGCAGGGTTGGGGTGC
CCCACCTGTGACGAGGTAGGGGTAGGGCGG
TGATGGCCAGGAAGATGCCCAGGAAGGTGA
GGGCCTGGTGTTCCCTTTAAGCGAAGGTGG
CTGTGGATGTTGTAGACCCTGGGATGGTGG
AGGCAAAACAGCAAATGAACCCAAAGGGGA
GCCGCCCTACCCCTACCTCGTCACAGGTGG
GCAAAACAGCAAATGAACCCAAAGGGGAAG
GTCGTAGGAGGCTGTCCTGTCTGCAGGTGA
GGCCGGCCAGGGCGACTACGCGTGCGGCCC
GGCCGCTCCTGCAGCAGCGGCTTGTGGTCC
GGCAAAACAGCAAATGAACCCAAAGGGGAA
CGCCTACAACCTGGAGGCCGGCCAGGGCGA
TGCGGGCCGCACGCGTAGTCGCCCTGGCCG
CCTGCAGCAGCGGCTTGTGGTCCATGGCGG
TCTGCTGCAGGGTTGGGGTGCTGGAGGACT
GCCGGCCTCCAGGTTGTAGGCGGGCGGCCG
CCGGCTGTGGATGTTGTAGACCCTGGGATG
CCATGGACCACAAGCCGCTGCTGCAGGAGC
TGTGACGAGGTAGGGGTAGGGCGGCGGCGG
TGGATGTTGTAGACCCTGGGATGGTGGGTG
AGGAGCGGCCGCCCGCCTACAACCTGGAGG
CTGGCCGGCCTCCAGGTTGTAGGCGGGCGG
GGATGTTGTAGACCCTGGGATGGTGGGTGG
ACGAGGTAGGGGTAGGGCGGCGGCGGGGGC
AGGATGATGGCCAGGAAGATGCCCAGGAAG
CTCTGCCCGTCTCTGCTGCAGGGTTGGGGT
GACGAGGTAGGGGTAGGGCGGCGGCGGGGG
GTTGTAGACCCTGGGATGGTGGGTGGGTAT
GGCGGGGATGGCGCCGTAGCCGTGCGGGCC
GGGTTCATTTGCTGTTTTGCCTTGAGGAAG
CGAGGTAGGGGTAGGGCGGCGGCGGGGGCG
CCTGGCCGGCCTCCAGGTTGTAGGCGGGCG
CTGGCCATCATCCTCTTCCCCTTTGGGTTC
TGAACCCAAAGGGGAAGAGGATGATGGCCA
GAGGTAGGGGTAGGGCGGCGGCGGGGGCGC
GCCGCCCGCCTACAACCTGGAGGCCGGCCA
GGCCGCACGCGTAGTCGCCCTGGCCGGCCT
TGTTGTAGACCCTGGGATGGTGGGTGGGTA
GGATAGCGGGTGACGGTCCGGCTGTGGATG
AACTGTGGAGCCACCTTCGCTTAAAGGGAA
CCTGGCCATCATCCTCTTCCCCTTTGGGTT
TTAAGCGAAGGTGGCTCCACAGTTGGGGCA
TCTGCCCGTCTCTGCTGCAGGGTTGGGGTG
ACTGTGGAGCCACCTTCGCTTAAAGGGAAC
TTTAAGCGAAGGTGGCTCCACAGTTGGGGC
GCGGGGATGGCGCCGTAGCCGTGCGGGCCG
CGCCCTGGCCGGCCTCCAGGTTGTAGGCGG
TCCGGCTGTGGATGTTGTAGACCCTGGGAT
GCGTAGTCGCCCTGGCCGGCCTCCAGGTTG
CCGCCTACAACCTGGAGGCCGGCCAGGGCG
GCTGGAGGACTGCTTCACCTTCCTGGGCAT
GCTTCACCTTCCTGGGCATCTTCCTGGCCA
GCGGCGGCGGGGGCGCGGCGGGGATGGCGC
GTCTCTGCTGCAGGGTTGGGGTGCTGGAGG
TAGGGCGGCGGCGGGGGCGCGGCGGGGATG
TGCTGGAGGACTGCTTCACCTTCCTGGGCA
GGTAGGGCGGCGGCGGGGGCGCGGCGGGGA
AGGGGTAGGGCGGCGGCGGGGGCGCGGCGG
GTAGGGCGGCGGCGGGGGCGCGGCGGGGAT;
or
(v) comprises any anti-BRI3 agent utilizing CRISPR/Cas9 with an inactivated endonuclease, referred to as “dead”Cas9 or “dCas9.”

9. The method according to any one of claims 1-7, wherein the active agent comprises or consists of one or more short-guide RNAs having sequences selected from one or more of oligonucleotide sequences AACTCTATCGTGGTCGTAGG, CGTCACAGGTGGGCCCGTAA, GACTACGCGTGCGGCCCGCA (depicted here in “sense” orientation) and optionally inactivated endonuclease, further optionally “dead”Cas9 or “dCas9.”

10. The method according to any one of claims 1-9, further comprising administering at least one other active agent, optionally wherein the at least one other active agent is levodopa, carbidopa, a dopamine agonist (e.g., pramipexole, ropinirole, rotigotine, apomorphine), a monoamine oxidase B (MAO B) inhibitor (e.g., selegiline, rasagiline, safinamide), a catechol O-methyltrasnferase (COMT) inhibitor (e.g., entacapone, tolcapone), an anticholinergic (e.g., benztropine), or amantadine, or any combination thereof, further optionally comprising administering deep brain stimulation (DBS).

11. The method according to any one of claims 1-10, further comprising any one or more of the following:
(i) increasing CD16⁺ monocytes;
(ii) increasing B cells;
(iii) increasing dendritic cells;
(iv) reducing CD14⁺ monocytes; and
(v) reducing CD4⁺ T cells,
optionally wherein the increasing and/or reducing in any one or more of (i)-(v) at least takes place in PBMCs.

12. The method according to any one of claims 1-11, further comprising detecting the expression and/or function of one or more of BRI3, FAM89B, PCBP1, ATP5F1E (or ATP5E), SH2B2, LMO2, TAF10, MAP2K2, TLE4, GRK6, RNF187, TNNT1, PTP4A2, SIAH2, YBX3, LAMP1, RPL8, EID2, CAPG, SRM, TMSB10, FYB, HLA-E, GLRX5, SEC61G, TMEM9, DBP, XRRA1, BLOC1S4, TUFM, HDDC2, EVL, UBB, RAC2, OAS1, LSP1, or ARF5, the expression of which is to be increased or decreased, wherein said detecting occurs prior, during and/or after said administrating, optionally wherein the detecting comprises detecting in one or more samples from the treated subject, optionally a blood sample and/or a brain sample.

13. A method of determining whether a subject has a neurodegenerative disease associated with neuroinflammation or whether a subject is at increased risk of developing a neurodegenerative disease associated with neuroinflammation, comprising:
(a) measuring the expression of BRI3, FAM89B, PCBP1, ATP5F1E (or ATP5E), SH2B2, LMO2, TAF10, MAP2K2, TLE4, GRK6, RNF187, TNNT1, PTP4A2, SIAH2, YBX3, LAMP1, RPL8, EID2, CAPG, SRM, TMSB10, FYB, HLA-E, GLRX5, SEC61G, TMEM9, DBP, XRRA1, BLOC1S4, TUFM, HDDC2, EVL, UBB, RAC2, OAS1, LSP1, or ARF5, or any combination thereof in the subject or in a sample from the subject, and optionally measuring the quantity (number and/or percentage) of CD16⁺ monocytes, B cells, dendritic cells, CD14⁺ monocytes, and CD4⁺ T cells in the sample; and
(b) determining that the subject has a neurodegenerative disease
if:
(i) the expression of BRI3, FAM89B, PCBP1, ATP5F1E (or ATP5E), SH2B2, LMO2, TAF10, MAP2K2, TLE4, GRK6, RNF187, TNNT1, PTP4A2, SIAH2, YBX3, LAMP1, RPL8, EID2, CAPG, SRM, TMSB10, FYB, HLA-E, GLRX5, SEC61G, TMEM9, DBP, XRRA1, BLOC1S4, or TUFM, or any combination thereof is higher than a normal control; and/or
(ii) the expression of HDDC2, EVL, UBB, RAC2, OAS1, LSP1, or ARF5, or any combination thereof is lower than a normal control,
and optionally if:
(iii) the quantity of CD16⁺ monocytes, B cells, and/or dendritic cells is reduced; and/or
(iv) the quantity of CD14⁺ monocytes and/or CD4⁺ T cells is increased,
optionally wherein the neurodegenerative disease a neurodegenerative disease associated with neuroinflammation is PD.

14. A method of determining whether a subject has inflammation associated with a neurodegenerative disease or is at risk of developing inflammation associated with a neurodegenerative disease, comprising:
(a) measuring the expression of BRI3, FAM89B, PCBP1, ATP5F1E (or ATP5E), SH2B2, LMO2, TAF10, MAP2K2, TLE4, GRK6, RNF187, TNNT1, PTP4A2, SIAH2, YBX3, LAMP1, RPL8, EID2, CAPG, SRM, TMSB10, FYB, HLA-E, GLRX5, SEC61G, TMEM9, DBP, XRRA1, BLOC1S4, TUFM, HDDC2, EVL, UBB, RAC2, OAS1, LSP1, or ARF5, or any combination thereof in the subject or in a sample from the subject, and optionally measuring the quantity (number and/or percentage) of CD16⁺ monocytes, B cells, dendritic cells, CD14⁺ monocytes, and CD4⁺ T cells in the sample; and
(b) determining that the subject has inflammation associated with a neurodegenerative disease
if:
(i) the expression of BRI3, FAM89B, PCBP1, ATP5F1E (or ATP5E), SH2B2, LMO2, TAF10, MAP2K2, TLE4, GRK6, RNF187, TNNT1, PTP4A2, SIAH2, YBX3, LAMP1, RPL8, EID2, CAPG, SRM, TMSB10, FYB, HLA-E, GLRX5, SEC61G, TMEM9, DBP, XRRA1, BLOC1S4, or TUFM, or any combination thereof is higher than a control; and/or
(ii) the expression of HDDC2, EVL, UBB, RAC2, OAS1, LSP1, or ARF5, or any combination thereof is lower than a control,
and optionally if:
(iii) the quantity of CD16⁺ monocytes, B cells, and/or dendritic cells is reduced; and/or
(iv) the quantity of CD14⁺ monocytes and/or CD4⁺ T cells is increased, optionally wherein the neurodegenerative disease is PD.

15. The method according to claim 13 or 14, comprising one or more of the following:
(I) in (a), at least the expression of BRI3 is measured;
(II) in (b), at least the expression of BRI3 is higher than the control;
(III) in (b) (i), the expression is higher than the control by about 25%, about 30%, about 40%, about 45%, about 50%, about 55% about 60%, about 65%, about 70%, about 75%, about 80%, about 85%, about 90%, about 95%, about 100%, about 150%, about 200%, about 300%, about 400%, about 500%, about 600%, about 800%, about 900%, about 1000%, about 2000%, about 3000%, about 4000%, about 5000%, or about 10000%; and/or
(IV) in (b) (ii), the expression is lower than the control by about 25%, about 30%, about 40%, about 45%, about 50%, about 55% about 60%, about 65%, about 70%, about 75%, about 80%, about 85%, about 90%, about 95%, or about 100%.

16. A method of determining whether the severity and/or stage of and/or inflammation associated with a neurodegenerative disease has increased in a subject, comprising:
(a) measuring the expression of BRI3, FAM89B, PCBP1, ATP5F1E (or ATP5E), SH2B2, LMO2, TAF10, MAP2K2, TLE4, GRK6, RNF187, TNNT1, PTP4A2, SIAH2, YBX3, LAMP1, RPL8, EID2, CAPG, SRM, TMSB10, FYB, HLA-E, GLRX5, SEC61G, TMEM9, DBP, XRRA1, BLOC1S4, TUFM, HDDC2, EVL, UBB, RAC2, OAS1, LSP1, or ARF5, or any combination thereof in the subject or in a sample from the subject at a first time point and a second time point, and optionally measuring the quantity (number and/or percentage) of CD16⁺ monocytes, B cells, dendritic cells, CD14⁺ monocytes, and CD4+ T cells in the subject or in the sample at the first time point and the second time point; and
(b) determining that the severity and/or stage and/or inflammation has increased
if:
(i) the expression of BRI3, FAM89B, PCBP1, ATP5F1E (or ATP5E), SH2B2, LMO2, TAF10, MAP2K2, TLE4, GRK6, RNF187, TNNT1, PTP4A2, SIAH2, YBX3, LAMP1, RPL8, EID2, CAPG, SRM, TMSB10, FYB, HLA-E, GLRX5, SEC61G, TMEM9, DBP, XRRA1, BLOC1S4, or TUFM, or any combination thereof is higher at the second time point than the first time point; and/or
(ii) the expression of HDDC2, EVL, UBB, RAC2, OAS1, LSP1, or ARF5, or any combination thereof is lower at the second time point than the first time point,
and optionally if:
(iii) the quantity of CD16⁺ monocytes, B cells, and/or dendritic cells is lower at the second time point than the first time point; and/or
(iv) the quantity of CD14⁺ monocytes and/or CD4⁺ T cells is higher at the second time point than the first time point,
optionally wherein the neurodegenerative disease is PD,
further optionally wherein the method comprises one or more of the following:
(I) in (a), at least the expression of BRI3 is measured;
(II) in (b), at least the expression of BRI3 is higher at the second time point than the first time point;
(III) in (b) (i), the expression is higher at the second time point than the first time point by about 25%, about 30%, about 40%, about 45%, about 50%, about 55% about 60%, about 65%, about 70%, about 75%, about 80%, about 85%, about 90%, about 95%, about 100%, about 150%, about 200%, about 300%, about 400%, about 500%, about 600%, about 800%, about 900%, about 1000%, about 2000%, about 3000%, about 4000%, about 5000%, or about 10000%;
(IV) in (b) (ii), the expression is lower at the second time point than the first time point by about 25%, about 30%, about 40%, about 45%, about 50%, about 55% about 60%, about 65%, about 70%, about 75%, about 80%, about 85%, about 90%, about 95%, or about 100%;
(V) in (b) (iii), the quantity is lower at the second time point than the first time point by about 25%, about 30%, about 40%, about 45%, about 50%, about 55% about 60%, about 65%, about 70%, about 75%, about 80%, about 85%, about 90%, about 95%, or about 100%; and/or
(VI) in (b) (iv), the quantity is higher at the second time point than the first time point by about 25%, about 30%, about 40%, about 45%, about 50%, about 55% about 60%, about 65%, about 70%, about 75%, about 80%, about 85%, about 90%, about 95%, about 100%, about 150%, about 200%, about 300%, about 400%, about 500%, about 600%, about 800%, about 900%, about 1000%, about 2000%, about 3000%, about 4000%, about 5000%, or about 10000%.

17. A method of determining whether a therapy or prophylaxis for a neurodegenerative disease is effective in a subject, comprising:
(a) measuring the expression of BRI3, FAM89B, PCBP1, ATP5F1E (or ATP5E), SH2B2, LMO2, TAF10, MAP2K2, TLE4, GRK6, RNF187, TNNT1, PTP4A2, SIAH2, YBX3, LAMP1, RPL8, EID2, CAPG, SRM, TMSB10, FYB, HLA-E, GLRX5, SEC61G, TMEM9, DBP, XRRA1, BLOC1S4, TUFM, HDDC2, EVL, UBB, RAC2, OAS1, LSP1, or ARF5, or any combination thereof in the subject or in a sample from the subject before and at one or more time points after starting the therapy, and optionally measuring the quantity (number and/or percentage) of CD16⁺ monocytes, B cells, dendritic cells, CD14⁺ monocytes, and CD4⁺ T cells in the subject or in the sample before and at one or more time points after starting the therapy; and
(b) determining that the therapy is effective
if:
(i) the expression of BRI3, FAM89B, PCBP1, ATP5F1E (or ATP5E), SH2B2, LMO2, TAF10, MAP2K2, TLE4, GRK6, RNF187, TNNT1, PTP4A2, SIAH2, YBX3, LAMP1, RPL8, EID2, CAPG, SRM, TMSB10, FYB, HLA-E, GLRX5, SEC61G, TMEM9, DBP, XRRA1, BLOC1S4, or TUFM, or any combination thereof is lower at least one time point after starting the therapy compared to before starting the therapy; and/or
(ii) the expression of HDDC2, EVL, UBB, RAC2, OAS1, LSP1, or ARF5, or any combination thereof is higher at least one time point after starting the therapy compared to before starting the therapy,
and optionally if:
(iii) the quantity of CD16+ monocytes, B cells, and/or dendritic cells is lower at least one time point after starting the therapy compared to before starting the therapy; and/or
(iv) the quantity of CD14⁺ monocytes and/or CD4⁺ T cells is higher least one time point after starting the therapy compared to before starting the therapy,
optionally wherein the neurodegenerative disease is PD,
further optionally wherein the method comprises one or more of the following:
(I) in (a), at least the expression of BRI3 is measured;
(II) in (b), at least the expression of BRI3 is lower at least one time point after starting the therapy compared to before starting the therapy;
(III) in (b) (i), the expression is lower at least one time point after starting the therapy compared to before starting the therapy by about 25%, about 30%, about 40%, about 45%, about 50%, about 55% about 60%, about 65%, about 70%, about 75%, about 80%, about 85%, about 90%, about 95%, or about 100%;
(IV) in (b) (ii), the expression is higher at least one time point after starting the therapy compared to before starting the therapy by about 25%, about 30%, about 40%, about 45%, about 50%, about 55% about 60%, about 65%, about 70%, about 75%, about 80%, about 85%, about 90%, about 95%, about 100%, about 150%, about 200%, about 300%, about 400%, about 500%, about 600%, about 800%, about 900%, about 1000%, about 2000%, about 3000%, about 4000%, about 5000%, or about 10000%;
(V) in (b) (iii), the quantity is higher at least one time point after starting the therapy compared to before starting the therapy by about 25%, about 30%, about 40%, about 45%, about 50%, about 55% about 60%, about 65%, about 70%, about 75%, about 80%, about 85%, about 90%, about 95%, about 100%, about 150%, about 200%, about 300%, about 400%, about 500%, about 600%, about 800%, about 900%, about 1000%, about 2000%, about 3000%, about 4000%, about 5000%, or about 10000%; and/or
(VI) in (b) (iv), the quantity is lower at least one time point after starting the therapy compared to before starting the therapy by about 25%, about 30%, about 40%, about 45%, about 50%, about 55% about 60%, about 65%, about 70%, about 75%, about 80%, about 85%, about 90%, about 95%, or about 100%.

18. The method according to any one of claims 13-17, wherein, in (a), measuring is via one or more of RNA sequencing, RNA-sequencing at single cell resolution, DNA array, flow cytometry, histochemistry, protein detection (optionally by use of bead-based or solid phase protein detection methods, further optionally wherein protein detection is effected by the use of one or more of an immunosorbent assay, gel electrophoresis, SDS-PAGE (polyacrylamide gel electrophoresis), Liquid chromatography-mass spectrometry (LC-MS), HPLC, ELISA, immunoelectrophoresis, immunostaining, Western blot, protein colorimetric assay, flow cytometry, electron microscopy, an enzyme assay, immune fluorescence, spectrophotometry, and the like) or imaging, optionally wherein the sample comprises PBMCs, monocytes, dendritic cells, and/or central nervous system (CNS) cells, optionally wherein the monocytes are circulating monocytes, further optionally wherein the monocytes are CD16⁺ monocytes, CD14⁺ monocytes, and/or meningeal monocytes, and yet further optionally wherein the CNS cells comprise microglia.

19. A method of screening for a therapeutic agent for a neurodegenerative disease, comprising:
(a) applying a candidate therapeutic agent to (1) one or more cells derived from a subject with the neurodegenerative disease, (2) one or more neurodegenerative disease cell line cells, or (3) a cell or tissue culture comprising a sample derived from a neurodegenerative disease patient;
(b) after step (a), measuring the expression of BRI3, FAM89B, PCBP1, ATP5F1E (or ATP5E), SH2B2, LMO2, TAF10, MAP2K2, TLE4, GRK6, RNF187, TNNT1, PTP4A2, SIAH2, YBX3, LAMP1, RPL8, EID2, CAPG, SRM, TMSB10, FYB, HLA-E, GLRX5, SEC61G, TMEM9, DBP, XRRA1, BLOC1S4, TUFM, HDDC2, EVL, UBB, RAC2, OAS1, LSP1, or ARF5, or any combination thereof in said (1), (2), or (3), and optionally measuring the quantity (number and/or percentage) of CD16⁺ monocytes, B cells, dendritic cells, CD14⁺ monocytes, and CD4⁺ T cells in said (1), (2), or (3); and
(c) determining that the candidate therapeutic agent is effective
if:
(i) the expression of BRI3, FAM89B, PCBP1, ATP5F1E (or ATP5E), SH2B2, LMO2, TAF10, MAP2K2, TLE4, GRK6, RNF187, TNNT1, PTP4A2, SIAH2, YBX3, LAMP1, RPL8, EID2, CAPG, SRM, TMSB10, FYB, HLA-E, GLRX5, SEC61G, TMEM9, DBP, XRRA1, BLOC1S4, or TUFM, or any combination thereof is downregulated compared to an untreated or placebo control; and/or
(ii) the expression of HDDC2, EVL, UBB, RAC2, OAS1, LSP1, or ARF5, or any combination thereof is upregulated compared to an untreated or placebo control,
and optionally if:
(iii) the quantity of CD16⁺ monocytes, B cells, and/or dendritic cells is lower compared to an untreated or placebo control; and/or
(iv) the quantity of CD14⁺ monocytes and/or CD4⁺ T cells is higher compared to an untreated or placebo control,
optionally wherein the neurodegenerative disease is PD,
further optionally wherein the method comprises one or more of the following:
(I) in (a), at least the expression of BRI3 is measured;
(II) in (b), at least the expression of BRI3 is downregulated compared to an untreated or placebo control;
(III) in (b) (i), the expression is downregulated compared to an untreated or placebo control by about 25%, about 30%, about 40%, about 45%, about 50%, about 55% about 60%, about 65%, about 70%, about 75%, about 80%, about 85%, about 90%, about 95%, or about 100%;
(IV) in (b) (ii), the expression is upregulated compared to an untreated or placebo control by about 25%, about 30%, about 40%, about 45%, about 50%, about 55% about 60%, about 65%, about 70%, about 75%, about 80%, about 85%, about 90%, about 95%, about 100%, about 150%, about 200%, about 300%, about 400%, about 500%, about 600%, about 800%, about 900%, about 1000%, about 2000%, about 3000%, about 4000%, about 5000%, or about 10000%;
(V) in (b) (iii), the quantity is higher compared to an untreated or placebo control by about 25%, about 30%, about 40%, about 45%, about 50%, about 55% about 60%, about 65%, about 70%, about 75%, about 80%, about 85%, about 90%, about 95%, about 100%, about 150%, about 200%, about 300%, about 400%, about 500%, about 600%, about 800%, about 900%, about 1000%, about 2000%, about 3000%, about 4000%, about 5000%, or about 10000%; and/or
(VI) in (b) (iv), the quantity is lower compared to an untreated or placebo control by about 25%, about 30%, about 40%, about 45%, about 50%, about 55% about 60%, about 65%, about 70%, about 75%, about 80%, about 85%, about 90%, about 95%, or about 100%.

20. The method according to claim 19, wherein:
(A) the one or more cells in (1) or (2) comprise a PBMC, a blood cell, an immune cell, a monocyte, a dendritic cell, and/or a central nervous system (CNS) cell, optionally wherein the monocyte is a circulating monocyte, further optionally wherein the monocyte is a CD16⁺ monocyte, a CD14⁺ monocyte, and/or a meningeal monocyte, and yet further optionally wherein the CNS cell comprises or is a microglia; and/or
(B) the cell or tissue culture comprising a sample derived from a neurodegenerative disease patient in (3) comprises an organoid or three-dimensional cell culture.

21. A method of predicting whether a subject with a neurodegenerative disease will respond to a therapy comprising:
(a) applying the therapy to (1) one or more cells derived from a subject with the neurodegenerative disease or (2) a cell or tissue culture comprising a sample derived from a neurodegenerative disease patient;
(b) after step (a), measuring the expression of BRI3, FAM89B, PCBP1, ATP5F1E (or ATP5E), SH2B2, LMO2, TAF10, MAP2K2, TLE4, GRK6, RNF187, TNNT1, PTP4A2, SIAH2, YBX3, LAMP1, RPL8, EID2, CAPG, SRM, TMSB10, FYB, HLA-E, GLRX5, SEC61G, TMEM9, DBP, XRRA1, BLOC1S4, TUFM, HDDC2, EVL, UBB, RAC2, OAS1, LSP1, or ARF5, or any combination thereof in said (1) or (2), and optionally measuring the quantity (number and/or percentage) of CD16⁺ monocytes, B cells, dendritic cells, CD14⁺ monocytes, and CD4⁺ T cells in said (1) or (2); and
(c) predicting that the subject will respond to the therapy
if:
(i) the expression of BRI3, FAM89B, PCBP1, ATP5F1E (or ATP5E), SH2B2, LMO2, TAF10, MAP2K2, TLE4, GRK6, RNF187, TNNT1, PTP4A2, SIAH2, YBX3, LAMP1, RPL8, EID2, CAPG, SRM, TMSB10, FYB, HLA-E, GLRX5, SEC61G, TMEM9, DBP, XRRA1, BLOC1S4, or TUFM, or any combination thereof is downregulated compared to an untreated or placebo control; and/or
(ii) the expression of HDDC2, EVL, UBB, RAC2, OAS1, LSP1, or ARF5, or any combination thereof is upregulated compared to an untreated or placebo control,
and optionally if:
(iii) the quantity of CD16⁺ monocytes, B cells, and/or dendritic cells is lower compared to an untreated or placebo control; and/or
(iv) the quantity of CD14⁺ monocytes and/or CD4⁺ T cells is higher compared to an untreated or placebo control,
optionally wherein the neurodegenerative disease is PD,
further optionally wherein the method comprises one or more of the following:
(I) in (a), at least the expression of BRI3 is measured;
(II) in (b), at least the expression of BRI3 is downregulated compared to an untreated or placebo control;
(III) in (b) (i), the expression is downregulated compared to an untreated or placebo control by about 25%, about 30%, about 40%, about 45%, about 50%, about 55% about 60%, about 65%, about 70%, about 75%, about 80%, about 85%, about 90%, about 95%, or about 100%;
(IV) in (b) (ii), the expression is upregulated compared to an untreated or placebo control by about 25%, about 30%, about 40%, about 45%, about 50%, about 55% about 60%, about 65%, about 70%, about 75%, about 80%, about 85%, about 90%, about 95%, about 100%, about 150%, about 200%, about 300%, about 400%, about 500%, about 600%, about 800%, about 900%, about 1000%, about 2000%, about 3000%, about 4000%, about 5000%, or about 10000%;
(V) in (b) (iii), the quantity is higher compared to an untreated or placebo control by about 25%, about 30%, about 40%, about 45%, about 50%, about 55% about 60%, about 65%, about 70%, about 75%, about 80%, about 85%, about 90%, about 95%, about 100%, about 150%, about 200%, about 300%, about 400%, about 500%, about 600%, about 800%, about 900%, about 1000%, about 2000%, about 3000%, about 4000%, about 5000%, or about 10000%; and/or
(VI) in (b) (iv), the quantity is lower compared to an untreated or placebo control by about 25%, about 30%, about 40%, about 45%, about 50%, about 55% about 60%, about 65%, about 70%, about 75%, about 80%, about 85%, about 90%, about 95%, or about 100%.

22. The method according to claim 21, wherein:
(A) the one or more cells in (1) comprise a PBMC, a blood cell, an immune cell, a monocyte, a dendritic cell, and/or a central nervous system (CNS) cell, optionally wherein the monocyte is a circulating monocyte, further optionally wherein the monocyte is a CD16⁺ monocyte, a CD14⁺ monocyte, and/or meningeal monocyte, and yet further optionally wherein the CNS cell comprises or is a microglia; and/or
(B) the cell or tissue culture comprising a sample derived from a neurodegenerative disease patient in (2) comprises an organoid or three-dimensional cell culture.

23. The method according to claim 21 or 22, further comprising:
(d) if in step (c) the subject is predicted to respond to the therapy, administering said therapy to the subject.

24. The method according to any one of claims 1-23, wherein the neurodegenerative diseases is Parkinson's disease (PD), Alzheimer's disease (AD), multiple sclerosis (MS), Lewy body disease or Lewy body dementia (LBD), multiple system atrophy (MSA), progressive supranuclear palsy (PSP), amyotrophic lateral sclerosis (ALS) or motor neurone diseases (MND), Huntington's Disease (HD), spinocerebellar ataxia (SCA), Friedreich's ataxia (FA), spinal muscular atrophy (SMA), or prion disease (e.g., Creutzfeldt-Jakob disease (CJD)), optionally wherein the neurodegenerative diseases is PD.

25. An agent for treating or preventing or slowing the onset of a neurodegenerative disease, selected from:
(I) one which modifies the expression or function of BRI3, FAM89B, PCBP1, ATP5F1E (or ATP5E), SH2B2, LMO2, TAF10, MAP2K2, TLE4, GRK6, RNF187, TNNT1, PTP4A2, SIAH2, YBX3, LAMP1, RPL8, EID2, CAPG, SRM, TMSB10, FYB, HLA-E, GLRX5, SEC61G, TMEM9, DBP, XRRA1, BLOC1S4, TUFM, HDDC2, EVL, UBB, RAC2, OAS1, LSP1, or ARF5, or any combination thereof;
(II) one which decreases the expression of BRI3, FAM89B, PCBP1, ATP5F1E (or ATP5E), SH2B2, LMO2, TAF10, MAP2K2, TLE4, GRK6, RNF187, TNNT1, PTP4A2, SIAH2, YBX3, LAMP1, RPL8, EID2, CAPG, SRM, TMSB10, FYB, HLA-E, GLRX5, SEC61G, TMEM9, DBP, XRRA1, BLOC1S4, or TUFM, or any combination thereof;
(III) one which suppresses, blocks, or inhibits the function of BRI3, FAM89B, PCBP1, ATP5F1E (or ATP5E), SH2B2, LMO2, TAF10, MAP2K2, TLE4, GRK6, RNF187, TNNT1, PTP4A2, SIAH2, YBX3, LAMP1, RPL8, EID2, CAPG, SRM, TMSB10, FYB, HLA-E, GLRX5, SEC61G, TMEM9, DBP, XRRA1, BLOC1S4, or TUFM, or any combination thereof;
(IV) one which decreases the expression or function of BRI3;
(V) one which increases the expression of HDDC2, EVL, UBB, RAC2, OAS1, LSP1, or ARF5, or any combination thereof;
(VI) one which enhances the function of HDDC2, EVL, UBB, RAC2, OAS1, LSP1, or ARF5, or any combination thereof; or
(VII) any combination of (I)-(VI)
optionally wherein the neurodegenerative disease is PD.

26. The agent according to claim 25, selected from a clustered Regularly Interspaced Short Palindromic Repeat (CRISPR)/Cas gene editing agent, a zinc-finger nuclease (ZFN) gene editing agent, a transcription activator-like effector nuclease (TALEN) gene editing agent, a transposase-based gene therapy, an siRNA, an shRNA, an miRNA, an aptamer, an antibody, an antigen-binding antibody fragment (e.g., scFv, Fab, Fab′, (Fab′)2), a chimeric antigen receptor (CAR)-expressing cell, a peptide, a small molecule, a polymer, an expression vector encoding a gene of interest, or any combination thereof.

27. The agent according to claim 25, wherein the active agent:
(i) comprises a CRISPR/Cas gene editing agent against BRI3;
(ii) comprises or consists of a short-guide RNA (sgRNA) selected from the oligonucleotide sequences AACTCTATCGTGGTCGTAGG, CGTCACAGGTGGGCCCGTAA, GACTACGCGTGCGGCCCGCA (depicted here in “sense” orientation),
(iii) comprises or consists of a sgRNA targeting BRI3 falling within or including any of the following genomic sequences: GAGGAAGCGACGATGCCCCAACTGTGGAGC, ACCACGATAGAGTTGGCAGGATAGCGGGTG, AGTTGGGGCATCGTCGCTTCCTCAAGGCAA, TTACGGGCCCACCTGTGACGAGGTAGGGGT, GCCCTACCCCTACCTCGTCACAGGTGGGCC, TCCTGCCAACTCTATCGTGGTCGTAGGAGG, CCCGCTATCCTGCCAACTCTATCGTGGTCG, GGGCGACTACGCGTGCGGCCCGCACGGCTA, GCCCACCTGTGACGAGGTAGGGGTAGGGCG, CCCAGGGTCTACAACATCCACAGCCGGACC, CCACGATAGAGTTGGCAGGATAGCGGGTGA, TTGGCAGGATAGCGGGTGACGGTCCGGCTG, CAGGGATACCCACCCACCATCCCAGGGTCT, CCTGGTGTTCCCTTTAAGCGAAGGTGGCTC (as annotated in Gene ID 25798, NM_015379.5, or identical BRI3 sequences in prior or future annotations),
(iv) comprises a sgRNA sequence selected from one comprising or consisting of any of the following nucleic acid sequences:
GAGGAAGCGACGATGCCCCAACTGTGGAGC
GAGGAAGCGACGATGCCCCAACTGTGGAGC
GAGGAAGCGACGATGCCCCAACTGTGGAGC
GAGGAAGCGACGATGCCCCAACTGTGGAGC
GAGGAAGCGACGATGCCCCAACTGTGGAGC
GAGGAAGCGACGATGCCCCAACTGTGGAGC
GAGGAAGCGACGATGCCCCAACTGTGGAGC
GAGGAAGCGACGATGCCCCAACTGTGGAGC
GAGGAAGCGACGATGCCCCAACTGTGGAGC
GAGGAAGCGACGATGCCCCAACTGTGGAGC
GAGGAAGCGACGATGCCCCAACTGTGGAGC
ACCACGATAGAGTTGGCAGGATAGCGGGTG
AGTTGGGGCATCGTCGCTTCCTCAAGGCAA
TTACGGGCCCACCTGTGACGAGGTAGGGGT
GCCCTACCCCTACCTCGTCACAGGTGGGCC
TCCTGCCAACTCTATCGTGGTCGTAGGAGG
CCCGCTATCCTGCCAACTCTATCGTGGTCG
GGGCGACTACGCGTGCGGCCCGCACGGCTA
GCCCACCTGTGACGAGGTAGGGGTAGGGCG
CCCAGGGTCTACAACATCCACAGCCGGACC
CCACGATAGAGTTGGCAGGATAGCGGGTGA
TTGGCAGGATAGCGGGTGACGGTCCGGCTG
CAGGGATACCCACCCACCATCCCAGGGTCT
CCTGGTGTTCCCTTTAAGCGAAGGTGGCTC
AGCGGCCGCCCGCCTACAACCTGGAGGCCG
ACAGGGATACCCACCCACCATCCCAGGGTC
TAAGCGAAGGTGGCTCCACAGTTGGGGCAT
CCAAAGGGGAAGAGGATGATGGCCAGGAAG
TGGGATGGTGGGTGGGTATCCCTGTGGGCG
CAGCAAATGAACCCAAAGGGGAAGAGGATG
TACGGGCCCACCTGTGACGAGGTAGGGGTA
CTACGCGTGCGGCCCGCACGGCTACGGCGC
GGCGGGCGGCCGCTCCTGCAGCAGCGGCTT
GACCACAAGCCGCTGCTGCAGGAGCGGCCG
CTGCCCGTCTCTGCTGCAGGGTTGGGGTGC
CCCACCTGTGACGAGGTAGGGGTAGGGCGG
TGATGGCCAGGAAGATGCCCAGGAAGGTGA
GGGCCTGGTGTTCCCTTTAAGCGAAGGTGG
CTGTGGATGTTGTAGACCCTGGGATGGTGG
AGGCAAAACAGCAAATGAACCCAAAGGGGA
GCCGCCCTACCCCTACCTCGTCACAGGTGG
GCAAAACAGCAAATGAACCCAAAGGGGAAG
GTCGTAGGAGGCTGTCCTGTCTGCAGGTGA
GGCCGGCCAGGGCGACTACGCGTGCGGCCC
GGCCGCTCCTGCAGCAGCGGCTTGTGGTCC
GGCAAAACAGCAAATGAACCCAAAGGGGAA
CGCCTACAACCTGGAGGCCGGCCAGGGCGA
TGCGGGCCGCACGCGTAGTCGCCCTGGCCG
CCTGCAGCAGCGGCTTGTGGTCCATGGCGG
TCTGCTGCAGGGTTGGGGTGCTGGAGGACT
GCCGGCCTCCAGGTTGTAGGCGGGCGGCCG
CCGGCTGTGGATGTTGTAGACCCTGGGATG
CCATGGACCACAAGCCGCTGCTGCAGGAGC
TGTGACGAGGTAGGGGTAGGGCGGCGGCGG
TGGATGTTGTAGACCCTGGGATGGTGGGTG
AGGAGCGGCCGCCCGCCTACAACCTGGAGG
CTGGCCGGCCTCCAGGTTGTAGGCGGGCGG
GGATGTTGTAGACCCTGGGATGGTGGGTGG
ACGAGGTAGGGGTAGGGCGGCGGCGGGGGC
AGGATGATGGCCAGGAAGATGCCCAGGAAG
CTCTGCCCGTCTCTGCTGCAGGGTTGGGGT
GACGAGGTAGGGGTAGGGCGGCGGCGGGGG
GTTGTAGACCCTGGGATGGTGGGTGGGTAT
GGCGGGGATGGCGCCGTAGCCGTGCGGGCC
GGGTTCATTTGCTGTTTTGCCTTGAGGAAG
CGAGGTAGGGGTAGGGCGGCGGCGGGGGCG
CCTGGCCGGCCTCCAGGTTGTAGGCGGGCG
CTGGCCATCATCCTCTTCCCCTTTGGGTTC
TGAACCCAAAGGGGAAGAGGATGATGGCCA
GAGGTAGGGGTAGGGCGGCGGCGGGGGCGC
GCCGCCCGCCTACAACCTGGAGGCCGGCCA
GGCCGCACGCGTAGTCGCCCTGGCCGGCCT
TGTTGTAGACCCTGGGATGGTGGGTGGGTA
GGATAGCGGGTGACGGTCCGGCTGTGGATG
AACTGTGGAGCCACCTTCGCTTAAAGGGAA
CCTGGCCATCATCCTCTTCCCCTTTGGGTT
TTAAGCGAAGGTGGCTCCACAGTTGGGGCA
TCTGCCCGTCTCTGCTGCAGGGTTGGGGTG
ACTGTGGAGCCACCTTCGCTTAAAGGGAAC
TTTAAGCGAAGGTGGCTCCACAGTTGGGGC
GCGGGGATGGCGCCGTAGCCGTGCGGGCCG
CGCCCTGGCCGGCCTCCAGGTTGTAGGCGG
TCCGGCTGTGGATGTTGTAGACCCTGGGAT
GCGTAGTCGCCCTGGCCGGCCTCCAGGTTG
CCGCCTACAACCTGGAGGCCGGCCAGGGCG
GCTGGAGGACTGCTTCACCTTCCTGGGCAT
GCTTCACCTTCCTGGGCATCTTCCTGGCCA
GCGGCGGCGGGGGCGCGGCGGGGATGGCGC
GTCTCTGCTGCAGGGTTGGGGTGCTGGAGG
TAGGGCGGCGGCGGGGGCGCGGCGGGGATG
TGCTGGAGGACTGCTTCACCTTCCTGGGCA
GGTAGGGCGGCGGCGGGGGCGCGGCGGGGA
AGGGGTAGGGCGGCGGCGGGGGCGCGGCGG
GTAGGGCGGCGGCGGGGGCGCGGCGGGGAT;
or a sequence possessing at least 80, 85, 90, 95, or 95-99% identity to any of the foregoing sequences; or it
(v) comprises any anti-BRI3 agent utilizing CRISPR/Cas9 with an inactivated endonuclease, referred to as “dead”Cas9 or “dCas9.”

28. The agent according to claim 25, which comprises or consists of one or more short-guide RNAs having sequences selected from one or more of oligonucleotide sequences AACTCTATCGTGGTCGTAGG, CGTCACAGGTGGGCCCGTAA, GACTACGCGTGCGGCCCGCA (depicted here in “sense” orientation) and optionally inactivated endonuclease, further optionally “dead”Cas9 or “dCas9.”

29. A composition for treating or preventing a neurodegenerative disease, comprising the agent according to any one of claims 25-28, optionally wherein the neurodegenerative disease is PD.

30. The agent according to any one of claims 25-28 or the composition according to claim 29, wherein the neurodegenerative disease is selected from Parkinson's disease (PD), Alzheimer's disease (AD), multiple sclerosis (MS), Lewy body disease or Lewy body dementia (LBD), progressive supranuclear palsy (PSP), amyotrophic lateral sclerosis (ALS) or motor neurone diseases (MND), Huntington's Disease (HD), spinocerebellar ataxia (SCA), Friedreich's ataxia (FA), spinal muscular atrophy (SMA), or prion disease (e.g., Creutzfeldt-Jakob disease (CJD)) or is PD.

30. A kit comprising:
(a) (1) one or more cells derived from a subject with a neurodegenerative disease, (2) one or more neurodegenerative disease cell line cells, or (3) a cell or tissue culture comprising a sample derived from a neurodegenerative disease patient; and
(b) at least one primer set or at least one antibody for detecting expression of BRI3, FAM89B, PCBP1, ATP5F1E (or ATP5E), SH2B2, LMO2, TAF10, MAP2K2, TLE4, GRK6, RNF187, TNNT1, PTP4A2, SIAH2, YBX3, LAMP1, RPL8, EID2, CAPG, SRM, TMSB10, FYB, HLA-E, GLRX5, SEC61G, TMEM9, DBP, XRRA1, BLOC1S4, TUFM, HDDC2, EVL, UBB, RAC2, OAS1, LSP1, or ARF5, or any combination thereof,
optionally wherein said at least one primer set or at least one antibody in (b) comprises a primer set or an antibody for detecting expression of BRI3,
further optionally wherein the kit is for screening a therapeutic agent for treating or preventing the neurodegenerative disease,
and still further optionally wherein the neurodegenerative disease is PD.

31. A kit comprising:
(a) at least one primer set or at least one antibody for detecting expression of BRI3, FAM89B, PCBP1, ATP5F1E (or ATP5E), SH2B2, LMO2, TAF10, MAP2K2, TLE4, GRK6, RNF187, TNNT1, PTP4A2, SIAH2, YBX3, LAMP1, RPL8, EID2, CAPG, SRM, TMSB10, FYB, HLA-E, GLRX5, SEC61G, TMEM9, DBP, XRRA1, BLOC1S4, TUFM, HDDC2, EVL, UBB, RAC2, OAS1, LSP1, or ARF5, or any combination thereof; and
(b) an instruction sheet,
optionally wherein said at least one primer set or at least one antibody in (a) comprises a primer set or an antibody for detecting expression of BRI3.

32. The kit according to claim 30 or 31, wherein the expression is detected via a chemiluminescent, fluorescent, and/or colorimetric assay.


SEQUENCE LISTING (87 sequences)

SEQ ID NO: 1 - DNA, 20 bp, Artificial Sequence
  short-guide sgRNA
aactctatcgtggtcgtagg

SEQ ID NO: 2 - DNA, 20 bp, Artificial Sequence
  short-guide sgRNA
cgtcacaggtgggcccgtaa

SEQ ID NO: 3 - DNA, 20 bp, Artificial Sequence
  short-guide sgRNA
gactacgcgtgcggcccgca

SEQ ID NO: 4 - DNA, 30 bp, Artificial Sequence
  sgRNA targeting BRI3
gaggaagcgacgatgccccaactgtggagc

SEQ ID NO: 5 - DNA, 30 bp, Artificial Sequence
  sgRNA targeting BRI3
accacgatagagttggcaggatagcgggtg

SEQ ID NO: 6 - DNA, 30 bp, Artificial Sequence
  sgRNA targeting BRI3
agttggggcatcgtcgcttcctcaaggcaa

SEQ ID NO: 7 - DNA, 30 bp, Artificial Sequence
  sgRNA targeting BRI3
ttacgggcccacctgtgacgaggtaggggt

SEQ ID NO: 8 - DNA, 30 bp, Artificial Sequence
  sgRNA targeting BRI3
gccctacccctacctcgtcacaggtgggcc

SEQ ID NO: 9 - DNA, 30 bp, Artificial Sequence
  sgRNA targeting BRI3
tcctgccaactctatcgtggtcgtaggagg

SEQ ID NO: 10 - DNA, 30 bp, Artificial Sequence
  sgRNA targeting BRI3
cccgctatcctgccaactctatcgtggtcg

SEQ ID NO: 11 - DNA, 30 bp, Artificial Sequence
  sgRNA targeting BRI3
gggcgactacgcgtgcggcccgcacggcta

SEQ ID NO: 12 - DNA, 30 bp, Artificial Sequence
  sgRNA targeting BRI3
gcccacctgtgacgaggtaggggtagggcg

SEQ ID NO: 13 - DNA, 30 bp, Artificial Sequence
  sgRNA targeting BRI3
cccagggtctacaacatccacagccggacc

SEQ ID NO: 14 - DNA, 30 bp, Artificial Sequence
  sgRNA targeting BRI3
ccacgatagagttggcaggatagcgggtga

SEQ ID NO: 15 - DNA, 30 bp, Artificial Sequence
  sgRNA targeting BRI3
ttggcaggatagcgggtgacggtccggctg

SEQ ID NO: 16 - DNA, 30 bp, Artificial Sequence
  sgRNA targeting BRI3
cagggatacccacccaccatcccagggtct

SEQ ID NO: 17 - DNA, 30 bp, Artificial Sequence
  sgRNA targeting BRI3
cctggtgttccctttaagcgaaggtggctc

SEQ ID NO: 18 - DNA, 30 bp, Artificial Sequence
  sgRNA
agcggccgcccgcctacaacctggaggccg

SEQ ID NO: 19 - DNA, 30 bp, Artificial Sequence
  sgRNA
acagggatacccacccaccatcccagggtc

SEQ ID NO: 20 - DNA, 30 bp, Artificial Sequence
  sgRNA
taagcgaaggtggctccacagttggggcat

SEQ ID NO: 21 - DNA, 30 bp, Artificial Sequence
  sgRNA
ccaaaggggaagaggatgatggccaggaag

SEQ ID NO: 22 - DNA, 30 bp, Artificial Sequence
  sgRNA
tgggatggtgggtgggtatccctgtgggcg

SEQ ID NO: 23 - DNA, 30 bp, Artificial Sequence
  sgRNA
cagcaaatgaacccaaaggggaagaggatg

SEQ ID NO: 24 - DNA, 30 bp, Artificial Sequence
  sgRNA
tacgggcccacctgtgacgaggtaggggta

SEQ ID NO: 25 - DNA, 30 bp, Artificial Sequence
  sgRNA
ctacgcgtgcggcccgcacggctacggcgc

SEQ ID NO: 26 - DNA, 30 bp, Artificial Sequence
  sgRNA
ggcgggcggccgctcctgcagcagcggctt

SEQ ID NO: 27 - DNA, 30 bp, Artificial Sequence
  sgRNA
gaccacaagccgctgctgcaggagcggccg

SEQ ID NO: 28 - DNA, 30 bp, Artificial Sequence
  sgRNA
ctgcccgtctctgctgcagggttggggtgc

SEQ ID NO: 29 - DNA, 30 bp, Artificial Sequence
  sgRNA
cccacctgtgacgaggtaggggtagggcgg

SEQ ID NO: 30 - DNA, 30 bp, Artificial Sequence
  sgRNA
tgatggccaggaagatgcccaggaaggtga

SEQ ID NO: 31 - DNA, 30 bp, Artificial Sequence
  sgRNA
gggcctggtgttccctttaagcgaaggtgg

SEQ ID NO: 32 - DNA, 30 bp, Artificial Sequence
  sgRNA
ctgtggatgttgtagaccctgggatggtgg

SEQ ID NO: 33 - DNA, 30 bp, Artificial Sequence
  sgRNA
aggcaaaacagcaaatgaacccaaagggga

SEQ ID NO: 34 - DNA, 30 bp, Artificial Sequence
  sgRNA
gccgccctacccctacctcgtcacaggtgg

SEQ ID NO: 35 - DNA, 30 bp, Artificial Sequence
  sgRNA
gcaaaacagcaaatgaacccaaaggggaag

SEQ ID NO: 36 - DNA, 30 bp, Artificial Sequence
  sgRNA
gtcgtaggaggctgtcctgtctgcaggtga

SEQ ID NO: 37 - DNA, 30 bp, Artificial Sequence
  sgRNA
ggccggccagggcgactacgcgtgcggccc

SEQ ID NO: 38 - DNA, 30 bp, Artificial Sequence
  sgRNA
ggccgctcctgcagcagcggcttgtggtcc

SEQ ID NO: 39 - DNA, 30 bp, Artificial Sequence
  sgRNA
ggcaaaacagcaaatgaacccaaaggggaa

SEQ ID NO: 40 - DNA, 30 bp, Artificial Sequence
  sgRNA
cgcctacaacctggaggccggccagggcga

SEQ ID NO: 41 - DNA, 30 bp, Artificial Sequence
  sgRNA
tgcgggccgcacgcgtagtcgccctggccg

SEQ ID NO: 42 - DNA, 30 bp, Artificial Sequence
  sgRNA
cctgcagcagcggcttgtggtccatggcgg

SEQ ID NO: 43 - DNA, 30 bp, Artificial Sequence
  sgRNA
tctgctgcagggttggggtgctggaggact

SEQ ID NO: 44 - DNA, 30 bp, Artificial Sequence
  sgRNA
gccggcctccaggttgtaggcgggcggccg

SEQ ID NO: 45 - DNA, 30 bp, Artificial Sequence
  sgRNA
ccggctgtggatgttgtagaccctgggatg

SEQ ID NO: 46 - DNA, 30 bp, Artificial Sequence
  sgRNA
ccatggaccacaagccgctgctgcaggagc

SEQ ID NO: 47 - DNA, 30 bp, Artificial Sequence
  sgRNA
tgtgacgaggtaggggtagggcggcggcgg

SEQ ID NO: 48 - DNA, 30 bp, Artificial Sequence
  sgRNA
tggatgttgtagaccctgggatggtgggtg

SEQ ID NO: 49 - DNA, 30 bp, Artificial Sequence
  sgRNA
aggagcggccgcccgcctacaacctggagg

SEQ ID NO: 50 - DNA, 30 bp, Artificial Sequence
  sgRNA
ctggccggcctccaggttgtaggcgggcgg

SEQ ID NO: 51 - DNA, 30 bp, Artificial Sequence
  sgRNA
ggatgttgtagaccctgggatggtgggtgg

SEQ ID NO: 52 - DNA, 30 bp, Artificial Sequence
  sgRNA
acgaggtaggggtagggcggcggcgggggc

SEQ ID NO: 53 - DNA, 30 bp, Artificial Sequence
  sgRNA
aggatgatggccaggaagatgcccaggaag

SEQ ID NO: 54 - DNA, 30 bp, Artificial Sequence
  sgRNA
ctctgcccgtctctgctgcagggttggggt

SEQ ID NO: 55 - DNA, 30 bp, Artificial Sequence
  sgRNA
gacgaggtaggggtagggcggcggcggggg

SEQ ID NO: 56 - DNA, 30 bp, Artificial Sequence
  sgRNA
gttgtagaccctgggatggtgggtgggtat

SEQ ID NO: 57 - DNA, 30 bp, Artificial Sequence
  sgRNA
ggcggggatggcgccgtagccgtgcgggcc

SEQ ID NO: 58 - DNA, 30 bp, Artificial Sequence
  sgRNA
gggttcatttgctgttttgccttgaggaag

SEQ ID NO: 59 - DNA, 30 bp, Artificial Sequence
  sgRNA
cgaggtaggggtagggcggcggcgggggcg

SEQ ID NO: 60 - DNA, 30 bp, Artificial Sequence
  sgRNA
cctggccggcctccaggttgtaggcgggcg

SEQ ID NO: 61 - DNA, 30 bp, Artificial Sequence
  sgRNA
ctggccatcatcctcttcccctttgggttc

SEQ ID NO: 62 - DNA, 30 bp, Artificial Sequence
  sgRNA
tgaacccaaaggggaagaggatgatggcca

SEQ ID NO: 63 - DNA, 30 bp, Artificial Sequence
  sgRNA
gaggtaggggtagggcggcggcgggggcgc

SEQ ID NO: 64 - DNA, 30 bp, Artificial Sequence
  sgRNA
gccgcccgcctacaacctggaggccggcca

SEQ ID NO: 65 - DNA, 30 bp, Artificial Sequence
  sgRNA
ggccgcacgcgtagtcgccctggccggcct

SEQ ID NO: 66 - DNA, 30 bp, Artificial Sequence
  sgRNA
tgttgtagaccctgggatggtgggtgggta

SEQ ID NO: 67 - DNA, 30 bp, Artificial Sequence
  sgRNA
ggatagcgggtgacggtccggctgtggatg

SEQ ID NO: 68 - DNA, 30 bp, Artificial Sequence
  sgRNA
aactgtggagccaccttcgcttaaagggaa

SEQ ID NO: 69 - DNA, 30 bp, Artificial Sequence
  sgRNA
cctggccatcatcctcttcccctttgggtt

SEQ ID NO: 70 - DNA, 30 bp, Artificial Sequence
  sgRNA
ttaagcgaaggtggctccacagttggggca

SEQ ID NO: 71 - DNA, 30 bp, Artificial Sequence
  sgRNA
tctgcccgtctctgctgcagggttggggtg

SEQ ID NO: 72 - DNA, 30 bp, Artificial Sequence
  sgRNA
actgtggagccaccttcgcttaaagggaac

SEQ ID NO: 73 - DNA, 30 bp, Artificial Sequence
  sgRNA
tttaagcgaaggtggctccacagttggggc

SEQ ID NO: 74 - DNA, 30 bp, Artificial Sequence
  sgRNA
gcggggatggcgccgtagccgtgcgggccg

SEQ ID NO: 75 - DNA, 30 bp, Artificial Sequence
  sgRNA
cgccctggccggcctccaggttgtaggcgg

SEQ ID NO: 76 - DNA, 30 bp, Artificial Sequence
  sgRNA
tccggctgtggatgttgtagaccctgggat

SEQ ID NO: 77 - DNA, 30 bp, Artificial Sequence
  sgRNA
gcgtagtcgccctggccggcctccaggttg

SEQ ID NO: 78 - DNA, 30 bp, Artificial Sequence
  sgRNA
ccgcctacaacctggaggccggccagggcg

SEQ ID NO: 79 - DNA, 30 bp, Artificial Sequence
  sgRNA
gctggaggactgcttcaccttcctgggcat

SEQ ID NO: 80 - DNA, 30 bp, Artificial Sequence
  sgRNA
gcttcaccttcctgggcatcttcctggcca

SEQ ID NO: 81 - DNA, 30 bp, Artificial Sequence
  sgRNA
gcggcggcgggggcgcggcggggatggcgc

SEQ ID NO: 82 - DNA, 30 bp, Artificial Sequence
  sgRNA
gtctctgctgcagggttggggtgctggagg

SEQ ID NO: 83 - DNA, 30 bp, Artificial Sequence
  sgRNA
tagggcggcggcgggggcgcggcggggatg

SEQ ID NO: 84 - DNA, 30 bp, Artificial Sequence
  sgRNA
tgctggaggactgcttcaccttcctgggca

SEQ ID NO: 85 - DNA, 30 bp, Artificial Sequence
  sgRNA
ggtagggcggcggcgggggcgcggcgggga

SEQ ID NO: 86 - DNA, 30 bp, Artificial Sequence
  sgRNA
aggggtagggcggcggcgggggcgcggcgg

SEQ ID NO: 87 - DNA, 30 bp, Artificial Sequence
  sgRNA
gtagggcggcggcgggggcgcggcggggat